(19) United States
(12) Patent Application Publication
(10) Pub. No.: US 2022/0306979 A1
(43) Pub. Date: Sep. 29, 2022

(54) METHOD FOR DETERMINING PROCESS VARIABLES IN CELL CULTIVATION PROCESSES

(71) Applicant: Hoffmann-La Roche Inc., Little Falls, NJ (US)

(72) Inventors: Kristina Erhard, Pöcking (DE); Tobias Grosskopf, München (DE); Wolfgang Paul, Penzberg (DE); Daniel Stefke, Geretsried (DE); Sriram Venkateswaran, Freiburg im Breisgau (DE)

(73) Assignee: Hoffmann-La Roche Inc., Little Falls, NJ (US)

(21) Appl. No.: 17/670,299

(22) Filed: Feb. 11, 2022

Related U.S. Application Data
(63) Continuation of application No. PCT/EP2020/072560, filed on Aug. 12, 2020.

(30) Foreign Application Priority Data
Aug. 14, 2019 (EP) 19191807.7

(51) Int. Cl. C12M 1/36; C12P 21/00; C12N 5/071; C12M 1/34; G06N 20/20

(52) U.S. Cl. CPC C12M 41/48; C12P 21/005; C12N 5/0682; C12M 41/46; C12M 41/12; C12M 41/30; G06N 20/20; C12N 2510/02

(57) ABSTRACT

High throughput cultivation systems are used in pharmaceutical research and development. In this connection, samples are taken and analyzed for important parameters using external analysis. The results of the analysis serve to assess the cultivation process and provide important information about the process. Especially with cultivations carried out in parallel, the manual effort of sample preparation is great and can lead to errors. In order to avoid the need for sampling and thus to minimize the errors, a method is described in the present patent application which makes desired target parameters accessible in the form of soft sensors by means of previously recorded process variables. Herein is described a method for determining process-relevant parameters in CHO processes (Chinese hamster ovary) in high-throughput cultivations, in particular glucose, lactate and the live cell density or the live cell volume.

### CROSS-REFERENCE TO RELATED APPLICATIONS

This application is a continuation of International Application No. PCT/EP2020/072560 having an International filing date of Aug. 12, 2020, which claims benefit of and priority to European Patent Application No. 19191807.7, filed Aug. 14, 2019, the contents of which are incorporated herein by reference in their entirety.

### FIELD OF INVENTION

The current invention is in the field of mammalian cell cultivation. More specifically, the object of the present invention is a method for the on-line determination of process target parameters based on historical on-line and off-line values of a set of process variables.

### TECHNICAL BACKGROUND

For the production of therapeutic agents in the pharmaceutical industry, the highest demands are placed on quality and reproducibility. For this reason, empirical standards (GMP guidelines, good manufacturing practice) are in place that define target values, process limits and deviations in order to meet these requirements. The US Food and Drug Administration (FDA) recently asked the pharmaceutical industry through a PAT (Process Analytical Technology) initiative to develop a better understanding of the processes carried out in order to increase the quality of their products [1]. In recent years, new technologies such as computer-based models have been used to advance the understanding of cell culture processes of, for example, CHO cells, which are used to produce therapeutic proteins.

Bioreactors are most often used to cultivate cells. Various process variables are recorded here during cultivation. These enable process monitoring as well as control and serve to maintain controlled environmental conditions. A distinction is made between on-line and off-line values. Both values provide important information about the process. On-line values are collected by appropriate sensors that are used for direct on-line control. Off-line values, however, are to be determined by manual sampling with subsequent, external analytical methods. Such off-line parameters are, for example, the live cell density, glucose and lactate concentrations. These can be used to assess the current cultivation conditions and, if necessary, to intervene in the regulation of the process.

The analysis of the samples requires an increased manual effort, especially in the case of high-throughput cultivation systems. These external methods can also lead to errors and device failures in some circumstances. In order to make the processes more efficient and robust, it is possible to use on-line values already recorded during the cultivation to obtain information on-line. In this way, the existing measured parameters and their relationships can be analyzed so as to be described using suitable mathematical models of machine learning.

Artificial neural networks (ANN) for monitoring biomass in fed-batch processes have been described [8]. Kroll et al. described a model-based soft sensor for determining the subpopulations of the CHO cell biomass [9].

Hutter, S., et al. discloses glycosylation flux analysis of immunoglobulin G in Chinese hamster ovary perfusion cell culture (Process 6 (2018) 176). They describes a metabolic flux analysis based approach to generate insights on glycosylation pathways. Hutter et al. are focusing on metabolic flux analysis in perfusion cell culture experiments. Only off-line determined parameters were used to fit a mechanistic (linear) model using a random forest model to rank the input parameters influence on the glycosylation outcome. As such, Hutter et al. disclose a statistical analysis based on off-line data and performed after the cultivation, i.e. a modeling tool to make (biological) sense of historic data. No predictive or on-line algorithm is disclosed.

In the White Paper “Biopharma PAT—Quality Attributes, Critical Process Parameters & Key Performance Indicators at the Bioreactor” (available at https://www.researchgate.net/publication/326804832_Biopharma_PAT_-_Quality_Attributes_Criticcal_Process_Parameters_Key_Performance_Indicators_at_the_Bioreactor) a high-level overview of process analytical technologies. The paper describes cultivation principles (e.g. batch, fed-batch and perfusion, monitoring methods). Therein impact of measurements like dissolved oxygen are used to gain process understanding. No prediction of any output parameters or any machine learning approaches are disclosed.

Rubin, J., et al., report that pH excursions impact CHO cell culture performance and antibody N-linked glycosylation (Bioprocess. Biosys. Eng., 41 (2018) 1731-1741). The authors disclose a study on the impact of cell culture pH on antibody glycosylation using typical off-line measurements of process parameters as conducted in any cultivation, and the influences of pH variation thereon.

Downey, B. J., et al. report a novel approach for using dielectric spectroscopy to predict viable cell volume (VCV) in early process development (Biotechnol. Prog. 30 (2014) 479-487).

Xiao, P., et al. reported the metabolic characterization of a CHO cell size increase phase in fed-batch cultures (Appl. Microbiol. Biotechnol. 101 (2017) 8101-8113).

Kroll, P., et al. reported about a soft sensor for monitoring biomass subpopulations in mammalian cell culture processes (Biotechnol. Lett. 39 (2017) 1667-1673). The authors used a turbidity physical sensor to measure viable cell counts (VCC, equivalent to VCD) based on a linear model.

### SUMMARY OF THE INVENTION

The present invention is based, at least in part, on the finding that by selecting certain, specific process variables from historical datasets, useful data-driven models can be obtained, which contain important parameters for the cultivation of CHO cells, such as, VCD (Viable Cell Density), VCV (Viable Cell Volume), glucose and lactate in real time.

With the method according to the invention, it becomes possible to provide precise online-like values for the target variables over the entire course of the cultivation without sampling.

The object of the invention is a method for determining the viable cell density, and/or the viable cell volume and/or the glucose concentration in the cultivation medium and/or the lactate concentration in the cultivation medium for and during the cultivation of a CHO cell expressing an antibody, using exclusively on-line measured values from said cultivation by means of a model for the cultivation of this CHO cell, characterized in that the model based on a feature matrix comprising the features ‘Time’, ‘CHT.PV’, ‘ACOT.PV’, ‘FED2T.PV’, ‘GEW.PV’, ‘CO2T.PV’, ‘ACO.PV’, ‘AO.PV’, ‘N2.PV’, ‘CO2.PV’, ‘FED3T.PV’, ‘OUR’ and ‘PH.PV’ is generated.

The features denote the parameters as follows:

| feature | variable/value | unit |
|---|---|---|
| ACO.PV | CO2 concentration in off-gas flow | Volume % |
| ACOT.PV | CO2 total in off-gas flow | Volume % |
| AO.PV | O2 concentration in off-gas flow | Volume % |
| CHT.PV | Temperature of cooling element | ° C. |
| CO2.PV | CO2 inflow | ml/min |
| CO2T.PV | CO2 inflow cumulative | ml |
| DRZ.PV | Revolutions of stirrer | rpm |
| FED2T.PV | Feed 2 cumulative | ml |
| FED3T.PV | Feed 3 cumulative | ml |
| GEW.PV | Fermenter weight | g |
| LGE.PV | Base addition cumulative | g |
| N2.PV | N2 inflow | ml/min |
| O2.PV | O2 inflow | ml/min |
| OUR | Oxygen utilization rate | mol/(l*h) |
| PH.PV | fermenter pH | — |
| PO2.PV | Oxygen concentration in fermenter liquid | % |
| | phase | Saturation |
| Time | Elapsed time since fermentation start | d |
| TEF.PV | Fermenter temperature | ° C. |

In one embodiment, the model is generated using the random forest method.

In one embodiment, the training dataset comprises at least 10 cultivation runs, preferably at least 60 cultivation runs.

In one embodiment, the model is obtained with a training dataset that includes cultivation runs of mammalian cells that express a complex IgG, i.e. an antibody that comprises a different form than a wild-type Y-shaped full length antibody, e.g. by comprising additional domains, such as one or more Fabs. In one embodiment, the training dataset also contains cultivation runs of mammalian cells that express standard IgG, i.e. a Y-shaped wild-type-like antibody without additional or deleted domains.

In one embodiment, approximately 80% of the datasets available for the model formation are used as training datasets and the remaining datasets are used as test datasets.

In one embodiment
a) the datasets available for the modeling are randomly divided into training and test datasets in a ratio of 80:20,
b) the model is formed,
c) the mean and the standard deviation for the determination of the target parameter for the datasets are determined from the training dataset and the mean and the standard deviation for the determination of the target parameter for the records are determined from the test dataset,
d) steps a) to c) are repeated until comparable, i.e. within at most 10%, preferably at most 5% of each other, mean values and standard deviations are achieved regarding the division between test and training datasets.

In one embodiment, missing data points in the datasets are supplemented by interpolation.

In one embodiment, the datasets contain a data point for at least 60 minutes, preferably a data point for approximately every 5 to 10 minutes.

### Specific Embodiments of the Invention

1. A method for determining one or more process variables during the cultivation of a mammalian cell, characterized in that
   the process variable(s) are determined solely
   i) by means of a data-driven model of the cultivation of the mammalian cell, which has been generated with a feature matrix comprising the process variables ‘Time’, ‘CHT.PV’, ‘ACOT.PV’, ‘FED2T.PV’, ‘GEW.PV’, ‘CO2T.PV’, ‘ACO.PV’, ‘AO.PV’, ‘N2.PV’, ‘LGE.PV’, ‘CO2.PV’, ‘FED3T.PV’, ‘OUR’ and ‘PH.PV’,
   and
   ii) by using solely/only on-line measured values from the cultivation.
2. The method according to embodiment 1, characterized in that the on-line measured values are used at least for the process variables ‘Time’, ‘CHT.PV’, ‘ACOT.PV’, ‘FED2T.PV’, ‘GEW.PV’, ‘CO2T. PV’, ‘ACO.PV’, ‘AO.PV’, ‘N2.PV’, ‘CO2.PV’, ‘FED3T.PV’, ‘OUR’ and ‘PH.PV’ of the cultivation.
3. A method for adjusting the glucose concentration to a target value during the cultivation of a mammalian cell, comprising the following steps
   a) determining the current values at least for the process variables ‘Time’, ‘CHT.PV’, ‘ACOT.PV’, ‘FED2T.PV’, ‘GEW.PV’, ‘CO2T.PV’, ‘ACO.PV’, ‘AO.PV’, ‘N2.PV’, ‘CO2.PV’, ‘FED3T.PV’, ‘OUR’ and ‘PH.PV’ of the cultivation,
   b) determining the current glucose concentration in the cultivation medium using the values as determined in a) by means of a data-driven model for the cultivation of the mammalian cell, which is generated using a feature matrix comprising the process variables ‘Time’, ‘CHT.PV’, ‘ACOT.PV’, ‘FED2T.PV’, ‘GEW.PV’, ‘CO2T.PV’, ‘ACO.PV’, ‘AO.PV’, ‘N2.PV’, ‘CO2.PV’, ‘FED3T.PV’, ‘OUR’ and ‘PH.PV’, and
   c) adding glucose until the target value is reached if the current glucose concentration as determined in b) is lower than the target value, and thereby adjusting the glucose concentration to a target value.
4. The method according to any one of embodiments 1 through 3, characterized in that the process variable(s) is/are selected from the group comprising the process variables viable cell density, viable cell volume, glucose concentration in the cultivation medium, and lactate concentration in the cultivation medium.
5. The method according to any one of embodiments 1 through 4, characterized in that the method is carried out without sampling and only on-line determined/measured values from said cultivation are used.
6. The method according to any one of embodiments 1 through 5, characterized in that the data-driven model is generated by means of machine learning.
7. The method according to any one of embodiments 1 through 6, characterized in that the data-driven model is generated using a method selected from the group comprising artificial neural networks and ensemble learning.
8. The method according to any one of embodiments 1 through 7, characterized in that the data-driven model is generated using the random forest method.
9. The method according to any one of embodiments 1 through 7, characterized in that the data-driven model is generated using the MLPRegressor method.
10. The method according to any one of embodiments 1 through 7, characterized in that the data-driven model is generated using the XGBoost method.
11. The method according to any one of embodiments 1 through 10, characterized in that the data-driven model is generated through supervised learning.
12. The method according to any one of embodiments 1 through 11, characterized in that the data-driven model is verified by cross-validation.
13. The method according to embodiment 12, characterized in that the cross-validation is a 10-fold cross-validation.
14. The method according to any one of embodiments 1 through 13, characterized in that the data-driven model is generated using a training dataset which comprises at least 10 cultivation runs.
15. The method according to embodiment 14, characterized in that the training dataset comprises at least 60 cultivation runs.
16. The method according to any one of embodiments 1 through 15, characterized in that approximately 80% of the datasets available for the model generation are used as training datasets and the remaining datasets are used as test datasets.
17. The method according to any one of embodiments 1 through 16, characterized in that
   a) the datasets available for modeling are randomly divided into training and test datasets in a ratio between 70:30 and 80:20,
   b) the model is formed,
   c) the mean value and the standard deviation for the determination of the process variable for the datasets from the training dataset is determined and the mean value and the standard deviation for the determination of the process variable for the datasets from the test dataset is determined,
   d) steps a) to c) are repeated until comparable mean values and standard deviations, i.e. within 10% of each other, preferably within 5% of each other, with respect to the test and training datasets are achieved, wherein the division obtained under a) is different for/with each new run.
18. The method according to any one of embodiments 1 through 17, characterized in that the datasets, which are used to generate the data-driven model, each contain the same number of data points.
19. The method according to any one of embodiments 1 through 18, characterized in that the data points in the datasets, which are used to generate the data-driven model, are each for the same time points of the cultivation.
20. The method according to any one of embodiments 1 through 19, characterized in that missing data points in the datasets are obtained by interpolation.
21. The method according to embodiment 20, characterized in that missing data points for glucose concentration and/or viable cell volume are obtained by a third degree polynomial fit.
22. The method according to any one of embodiments 20 through 21, characterized in that missing data points for lactate concentration are obtained by univariate spline fit.
23. The method according to any one of embodiments 20 through 22, characterized in that missing data points for viable cell density are obtained by Peleg fit.
24. The method according to any one of embodiments 1 through 23, characterized in that each dataset contains a data point at least every 144 minutes.
25. The method according to any one of embodiments 1 through 24, characterized in that each dataset contains a data point at least every 60 minutes.
26. The method according to any one of embodiments 1 through 25, characterized in that each dataset contains a data point approximately every 5 to 10 minutes.
27. The method according to any one of embodiments 1 through 26, characterized in that the mammalian cell is a CHO cell.
28. The method according to any one of embodiments 1 through 27, characterized in that the mammalian cell is a CHO-K1 cell.
29. The method according to any one of the embodiments 1 through 28, characterized in that the mammalian cell expresses and secretes a therapeutic protein.
30. The method according to any one of embodiments 1 through 29, characterized in that the mammalian cell expresses and secretes an antibody.
31. The method according to embodiment 30, characterized in that the antibody is a monoclonal and/or therapeutic antibody.
32. The method according to any one of embodiments 30 through 31, characterized in that the antibody is not a standard IgG antibody, i.e. is a wild-type, four chain, full-length antibody or is a complex antibody, i.e. an antibody comprising additional antibody and/or non-antibody domains compared to a standard antibody.
33. The method according to any one of embodiments 1 through 32, characterized in that the data-driven model is generated with a training dataset, which contains only complex IgG cultivation runs.
34. The method according to any one of embodiments 1 through 33, characterized in that the data-driven model is generated with a training dataset, which also contains standard IgG cultivation runs.
35. The method according to any one of embodiments 1 through 34, characterized in that the mammalian cell expresses and secretes a complex or standard IgG.
36. The method according to any one of embodiments 1 through 35, characterized in that the cultivation volume is 300 mL or less.
37. The method according to any one of embodiments 1 through 36, characterized in that the cultivation volume is 250 mL or less, 200 mL or less, 100 mL or less, 75 mL or less, between 200 and 250 mL, or between 50 and 100 mL.
38. The method according to any one of embodiments 1 through 37, characterized in that the cultivation is a fed-batch cultivation.
39. The method according to any one of embodiments 1 through 38, characterized in that the cultivation is carried out in a stirred-tank reactor.
40. The method according to any one of embodiments 1 through 39, characterized in that there is submerged gassing in the cultivation.
41. The method according to any one of embodiments 1 through 40, characterized in that the cultivation is carried out in a single-use bioreactor (SUB).
42. The method according to any one of embodiments 1 through 41, characterized in that the mammalian cell is cultivated in suspension or that the mammalian cell is a suspension growing mammalian cell.
43. The method according to any one of embodiments 1 through 42, characterized in that the data-driven model is generated by regression analysis.
44. Use of the viable cell volume as a target parameter in the generation of a data-driven model for determining process variables for the cultivation of mammalian cells in a volume of 300 mL or less.
45. The use according to embodiment 44, characterized in that the process variable(s) is/are selected from the group comprising the process variables viable cell density, viable cell volume, glucose concentration in the cultivation medium, and lactate concentration in the cultivation medium.
46. The use according to any one of embodiments 44 through 45, characterized in that the cultivation is carried out without sampling.
47. The use according to any one of embodiments 44 through 46, characterized in that the mammalian cell is a CHO cell.
48. The use according to any one of embodiments 44 through 47, characterized in that the mammalian cell is a CHO-K1 cell.
49. The use according to any one of embodiments 44 through 48, characterized in that the mammalian cell expresses and secretes a therapeutic protein.
50. The use according to any one of embodiments 44 through 49, characterized in that the mammalian cell expresses and secretes an antibody.
51. The use according to embodiment 50, characterized in that the antibody is a monoclonal and/or therapeutic antibody.
52. The use according to any one of embodiments 50 through 51, characterized in that the antibody is not a standard IgG antibody or is a complex antibody.
53. The use according to any one of embodiments 44 through 52, characterized in that the data-driven model is generated with a training dataset, which contains only complex IgG cultivation runs.
54. The use according to any one of embodiments 44 through 53, characterized in that the data-driven model is generated with a training dataset, which also contains standard IgG cultivation runs.
55. The use according to any one of embodiments 44 through 54, characterized in that the mammalian cell expresses and secretes a complex or standard IgG.

### DETAILED DESCRIPTION OF EMBODIMENTS OF THE INVENTION

In order to be able to achieve a high throughput of test cultivations, especially for complex molecules and molecular formats, the cultivation containers must be reduced in size and the cultivations must be automated. The success of a cultivation depends on the controlled process variables and the desired molecule can only be produced in high yields when optimal cultivation conditions are provided. Thus, fast and efficient control of the relevant process variables is required in order to be able to set the respective process variables and maintain optimal cultivation conditions. Such a control is particularly necessary for small-scale parallel cultivations, as each cultivation has to be monitored separately. In particular, the so called off-line process variables are a problem here, because on the one hand the required sampling and separate analysis result in a time offset, i.e. the cultivation continues and the process variables once determined off-line differ from the real process variables, and on the other hand the number of sampling points is significantly smaller compared to on-line available process variable, which leads to temporally worse control of this process variable.

It is therefore an object of the present invention to make process variables, which cannot be determined on-line but only off-line, especially due to the size of the employed cultivation vessel, available like process variables, which are available on-line, in the cultivation scale used, based on a data-driven model and in real time.

To produce recombinant proteins, bioreactors are mostly operated using a fed-batch process [4]. In addition to the fed-batch process, there are other operating modes such as the batch process and the continuous cultivation mode.

The fed-batch or feed process is one of the partially open systems. Advantages of this process are that nutrients such as glucose, glutamine and other amino acids can be added to the cultivation during the process. Resulting substrate limitations can be avoided and longer process times can be ensured. The substrates can be added continuously or in the form of (one or more) concentrated boluses. A suitable feeding strategy can be used to better control inhibitory effects and the accumulation of toxic by-products. However, this requires sufficient knowledge as well as control of the process.

To provide and maintain optimum conditions during the cultivation of mammalian cells, such as CHO cells, bioreactors are used almost exclusively [2]. The bioreactors used are mostly stirred-tank reactors. The cultivation takes place in suspension, i.e. of suspension growing cells.

Aerobic mammalian cells, such as CHO cells, require oxygen to maintain their cell metabolism. The cells are usually supplied with oxygen by submerged gassing of the culture broth. The concentration of dissolved oxygen in the reactor is one of the most important parameters for the cultivation of aerobic cells. The concentration of oxygen dissolved in the medium is determined by a number of transport resistances. Diffusion causes the oxygen to be transported from the gas bubble to the cell so that it can ultimately be metabolized by the cells. It is described that the transport mechanism can be carried out using the oxygen transport rate (Oxygen Transfer Rate, abbreviated OTR), whereas the consumption of oxygen by the cells themselves can be determined using the oxygen consumption rate (Oxygen Uptake Rate, abbreviated OUR) [2]. Appropriate exhaust gas analysis can provide the data required to calculate the OUR and OTR. Process variables such as temperature, pH value and the concentration of dissolved oxygen are monitored with suitable sensors and are comprised in the parameters to be controlled within a cultivation. These process variables have a significant influence on the effective productivity of mammalian cell lines [3].

In order to shorten development and set-up times for bioreactors, research and development are increasingly focused on single use technology (single use bioreactor; abbreviated: SUB). The great advantage of these systems is that complex cleaning processes and the necessary complex and costly cleaning methods such as CIP (cleaning in place) and SIP (sterilization in place) are not required.

Automated high-throughput cultivation systems such as the ambr250 system (automated microscale bioreactor) help accelerate drug development. Twelve single-use bioreactors with a volume of 250 mL each are available within this system. An automated liquid handler is used for pipetting and sampling. Operations are controlled by central process software. The entire ambr250 system is located under a laminar flow box to ensure a sterile environment during operation.

Soft sensors have been used more and more industrially in the past two decades for the monitoring of process variables [6]. Said process variables can usually only be determined with high analytical effort or externally, i.e. off-line. Particularly, when employing single-use systems on a small scale, the required additional sensors often cannot be installed (space and availability or connectivity to the disposable bioreactor, possibly not gamma-irradiatable, etc.). Therefore, there is a lack of continuous data of important process variables, especially at small cultivation scale, which could be used for process monitoring and which would allow regulation of said process variables, i.e. process target parameters. The name “soft sensor” combines the two terms “software” and “sensor”. The term “software” signifies computer-aided programming of a model. The output of these models provides information about the cultivation, in particular real-time values of process variables that would otherwise not be available due to the lack of the respective physical sensors [5].

Basically, soft sensors can be divided into two classes, model-driven and data-driven soft sensors.

Model-driven soft sensors are subject to theoretical process models. These require detailed knowledge of the ongoing process and describe said process using differential equations of state. This means that dynamic behavior of a process has to be described using mechanistic models. Such models are primarily developed for the planning and design of process plants and focus on the description of ideal equilibrium states.

In data-driven soft sensors (what are termed black box models), models based on machine learning are used. These include empirical models, which use historical data to describe process variable correlations. Biological processes are complex and not yet fully understood with regard to each and any aspect of the metabolism of the cultivated mammalian cells.

The field of application of data-driven soft sensors within the pharmaceutical industry is large. As a rule, cultivations are monitored and recorded.

It has now been found by the current inventors that such historical data can be used to generate data-driven models for on-line estimation of off-line process variables.

Process variables are primarily determined, i.e., made available, in real time; they can normally only be determined with difficulty and with increased analytical effort and the associated time offset. In addition, a robust and long-term stable on-line sensor system is not always available for the on-line monitoring of some process variables, such as biomass or certain substrate and product concentrations [7]. Although these parameters comprise important information about the cultivation process, they are only available at the limited time-points during the cultivation, i.e. at those at which a sample is taken and analyzed off-line.

With small systems, such as the ambr250 systems, it is not possible to measure certain process variables, such as turbidity and/or conductivity, due to the lack of probe ports. Furthermore, due to their design, some common probes require a relatively large amount of space, which is not available in these low volume systems.

Machine learning is the application of algorithms to describe the basic structures of datasets. Machine learning can be divided into two parts: supervised and unsupervised learning.

Supervised learning is used when a model is prepared to make predictions for future or unknown data based on training data. It is supervised because the training dataset already contains information about the desired output value. One example is the filtering of spam emails [10]: Accordingly, the algorithm receives a dataset which consists of spam and non-spam messages, and which already contains the information about spam/non-spam with which it goes through the learning phase. With a new, unmarked email, the algorithm now tries to predict what type of message it is. Since this is a categorical target variable (spam/non-spam), the term “classification” is used.

In the case of unsupervised learning, an attempt is made to acquire relationships within the dataset but without presenting a target variable to the algorithm. The focus is on exploring the underlying structure of the data in order to extract meaningful information therefrom. The simplest example of this group is clustering. In this exploratory data analysis, an attempt is made to divide the dataset into meaningful subgroups without prior knowledge of the actual group membership.

If the target variable is a continuous variable, one speaks of regression or regression analysis. Variables that are used to describe a regression model are called independent or explanatory variables. Based on this, an attempt is made to find a mathematical relationship between input variables and target parameter in order to be able to predict results.

The method according to the present invention uses supervised learning, in which the target variable is described by means of regression.

The modeling can be aligned schematically in the steps of: preprocessing, learning, evaluating and estimating of the target variables.

The preprocessing of the data is necessary to ensure that the model is able to correctly interpret the information it is based on. The dataset is prepared in the form of a feature matrix x and contains m features (columns) and n rows, which thus represent the explanatory variables. Each row n contains the specification of a feature for a specific data point.

$$X = [ \begin{matrix}{x}_{1}^{( 1 )} & {x}_{2}^{( 1 )} & … & {x}_{m}^{( 1 )} \\ {x}_{1}^{( 2 )} & {x}_{2}^{( 2 )} & … & {x}_{m}^{( 2 )} \\ ⋮ & ⋮ & ⋱ & ⋮ \\ {x}_{1}^{( n )} & {x}_{2}^{( n )} & … & {x}_{m}^{( n )}\end{matrix} ] ;$$

$$y = [ \begin{matrix}{y}^{( 1 )} \\ {y}^{( 2 )} \\ ⋮ \\ {y}^{( n )}\end{matrix} ]$$

The target variables are arranged in a vector y. Each row of the feature matrix x⁽ⁿ⁾ therefore contains the information for the associated value of the target variable y⁽ⁿ⁾.

Statistical analyses are used to identify suitable features. Once a suitable feature has been identified place and a corresponding feature matrix has been created, a subset (70-80% of the entire dataset) is made available to the model for learning. This subset is called the training dataset.

Typical data preprocessing might include providing the models with datasets in a standardized form. The data of each feature is thus given the property of a standard normal distribution with a mean of 0 and a standard deviation of 1. This increases the comparability of the features with each other and enables the learning algorithms to achieve their optimal performance [10].

Learning is the central part of model building. During learning, the model tries to understand and recognize the relationships between the data. Each model is subject to a mathematical formula with specific parameters. These adapt within the training process in order to describe the relationships between the data as good as possible.

Some models, such as neural networks, have other parameters that are not changed during the learning process. These are called hyperparameters. They influence the complexity of the models or the speed of the learning process and are determined before the training process. There is no fixed formula for choosing the right hyperparameters. Different models are therefore trained with different hyperparameters and then tested. Only then can it be judged which model is most suitable.

Randomized and raster-based algorithms are used to search for the optimal combination of hyperparameters. Each hyperparameter is represented by a list with different values. Models are trained in a grid search (GridSearch) with every possible combination from the respective lists. The required computing effort can be reduced by randomized searches. Various random parameter combinations are used, wherein the computing effort can be predetermined here. In one embodiment, the model is first executed with a randomized search for a rough estimate of the hyperparameters and then a grid search is carried out for the fine adjustment of the hyperparameters. The aim of learning is to train models so that bias and variance are kept as low as possible.

Models often learn the relationships between the training data better than with subsequent prediction with an unknown dataset. This behavior is called over-fitting. Accordingly, the model has memorized the training dataset and describes with new data the relationships with insufficient accuracy. Similar behavior can also be attributed to an excessive variance. Here the model uses too many input parameters for the dataset to be trained, leading to a complex model that only fits this dataset with a high data variance. Accordingly, the model learned the noise of the data without being able to map the actual relationship.

If, on the other hand, a model is not complex enough to be able to react to changes in the test dataset, this is called under-fitting. Then the bias is too great and the model can only imprecisely map the relationships of the training data to the test data.

Already during learning, k-fold cross-validations of the training dataset offer the possibility to avoid over-fitting of the models [11]. The training dataset is divided into k subsets. Then, k−1 subsets are used to train the model and the remaining subset is used as the test dataset. This procedure is repeated k times. In this way, k models are trained and k estimates of the target variable are obtained.

A performance estimate E of the model is generated for each run. As a performance estimate for regression, for example, the mean square deviation as a measure of the error is used. In practice, 10-fold cross-validations have proven to be a good compromise for bias and variance in most cases [12]:

$$E = \frac{1}{10} {\sum }_{l = 1}^{10} {E}_{i} .$$

Artificial neural networks (ANN) were described in 1943 by Warren McCulloch and Walter Pitts with a mathematical model of the neuron. Information transfer in biological systems could be made understandable in this way [13]. Frank Rosenblatt was then able to link the McCulloch-Pitts model of an artificial neuron with a learning rule and, thus, described the perceptron [14]. The perceptron still forms the basis of an ANN.

A simple perceptron has n inputs x₁, . . . , xₙ∈IR, each with a weighting w₁, . . . , wₙ∈IR. The output is represented by o∈IR. The processing of the input signals with appropriate weighting is the propagation function (input function) σ,

$$σ = {\sum }_{i = 1}^{n} {x}_{i} {w}_{i} ,$$

which describes the network input of a neuron. Via the activation function ϕ,

o=ϕ(σ),

the output o of a perceptron is then determined. Various functions can be used for ϕ, which can lead to activation of the perceptron.

The activation function, thus, calculates how strongly a neuron is activated depending on the threshold value and the network input [15]. If several of these neurons are interconnected in a suitable structure, the complex relationships between the input layer and the output layer can be mapped. The simplest form of such structural interconnections of simple neurons are feed-forward networks. These are arranged in layers and consist of an input layer, an output layer and, depending on the structure, several hidden layers.

In feed-forward networks (so called multilayer perceptrons), every neuron in one layer is connected to all other neurons in the next layer. These networks, thus, propagate the information content created through the network in a forward direction. Each neuron weights the incoming signal with an initially randomly selected weight and adds a bias term. The output of this neuron corresponds to the sum of all weighted input data. Depending on the number of neurons in a layer and the number of hidden layers, the complexity of the neural network can be determined.

Multi-layered feed-forward networks, which contain error feedback (backpropagation), are mostly used in supervised learning by an ANN [16].

The training of such a neural network can be divided into three steps:
Step 1: Feed-forward;
Step 2: Error calculation;
Step 3: Backpropagation.

In the first step, an input is made to the input layer of the network, which is propagated layer by layer through the network until there is an output from the network. The output of the network is compared with the expected value in the second step and the error of the network is calculated using an error function. Depending on the current weighting, each neuron within the hidden layers contributes to the calculated error to different extents. In the third step, the error is propagated backwards through the network, wherein the weighting is adjusted depending on the contribution of the weighting of an individual neuron to the error. The aim of the backpropagation algorithm is to minimize the error and usually uses a gradient descent method [17]. With this method, the quadratic distance between the output of the network and the expected output is calculated as an error function:

$$Err = \frac{1}{2} \sum {( true v alue - network output )}^{2}$$

In order to calculate the contribution of the weighting of each neuron to the error, the error function Err must be derived from the considered weight wᵢⱼ. Accordingly, only activation functions that are continuous and differentiable can be used here [17]. This determines the weight adjustment delta that will be used in the next iteration step. The relationship can be described mathematically as follows:

$$Δ {w}_{ij} = - η \frac{\partial Err}{\partial {w}_{ij}} .$$

The learning rate together with the number of iterations, is a hyperparameter that is established before training the model. The two steps are repeated until the maximum number of iterations or a defined error value has been reached and a good result can be achieved for an unknown input.

In addition, the random forest (RF) algorithm can be used in machine learning for regression problems [18]. RF learns through a multitude of decision trees and thus belongs to the category of ensemble learners. A decision tree can spread out from the root (top node, no predecessor). Each node divides the dataset into two groups based on a feature. The successors of the root can be leaves (no successors) or nodes (at least one successor). Nodes and leaves are connected by an edge. In the case of regression problems, [19]
a feature is assigned to each inner node (including the root);
a specific value of the target variable to be predicted is assigned to each leaf of the decision tree;
to each edge, a relation is assigned to a threshold value.

In a preferred embodiment, the RF uses the bagging principle (bootstrap aggregation principle) according to Breiman [18] to create a suitable training set, wherein the training set is created by a sampling from the entire training dataset with replacement. Some data may be selected multiple times, while other data are not selected as training data. The quantity of the training set always corresponds to the quantity of the entire training dataset. Each selected training set is used to generate a decision using a decision tree (classifier). The decisions of all training sets are then averaged, whereby a majority decision determines the final classification. The generation of the bootstrap samples thus creates a low correlation between the individual classifiers. Furthermore, the variance of the individual classifiers can be reduced and the overall classification performance increased [18].

In a preferred embodiment, a feature is used for the decision of a split (division of a node) during the creation of the tree, which feature makes the clearest decision regarding a random selection of features of the dataset. The selected split is no longer selected as the best split in terms of all features, but only the best within a random selection of the features. As a result of this randomization, the bias (distortion, systematic error) of the tree increases in the course of the creation. Because of the formation of the mean value of all trees contained in the RF, the variance decreases. The decreasing variance has a greater added value than the increase in the bias, which leads to an increased accuracy of the model [20].

Furthermore, overfitting of the models is almost prevented in RF predictions, since the average of all individual decisions is always considered [18].

The XGBoost (eXtreme Gradient BOOSTing) uses an ensemble of regression trees as a model-forming basis. Both the bagging principle already described and a special boosting technique is used to train the ensemble for the most accurate prediction possible. In simple terms, the boosting technique can be seen as a combination of a gradient descent method that consists of many weak learners [21]. These weak learners are usually no more precise than random guessing and are grouped together as strong learners in the course of creating the ensemble. A typical example of such a weak learner is a simple regression tree with only one node. The principle of the boosting algorithm is to select training data that are difficult to classify in order to learn from these poorly classified objects with these weak learners and, thus, improve the performance of the ensemble. Due to the complexity of the XGBoost, the algorithm is considered a black box. However, due to its scalability and speed of solving problems, the algorithm is used very successfully in a direct comparison of different models of machine learning [22].

The method implemented by XGBoost combines a gradient descent method with a boosting technique, which is described below using the original literature by Tianqi Chen, “XGBoost: A Scalable Tree Boosting System” [22].

With an ensemble that consists of K decision trees, the model can be described according to:

$${\sum }_{k = 1}^{K} {f}_{k} ,$$

wherein fₖ is the prediction of a single decision tree. Seen across all decision trees, a prediction can be made

$${\hat{y}}_{i} = {\sum }_{K}^{k = 1} {f}_{k} ( {x}_{i} )$$

wherein xᵢ is the feature vector for the i-th data point. In order to train the model, a loss function L is optimized. In the case of regression problems, the RMSE (root mean squared error) is used:

$$L = \frac{1}{N} {\sum }_{i = 1}^{N} {( {y}_{i} - {\hat{y}}_{i} )}^{2}$$

Regularization is an important part that prevents overfitting of the models:

$$Ω = γ T + \frac{1}{2} λ {\sum }_{j = 1}^{T} {w}_{j}^{2}$$

wherein T is the number of leaves, and w²ⱼ is the achieved scoring of the j-th leaf. If regularization and loss function are brought together, the basic objective function of the model can be formulated as

Obj=L+Ω,

wherein the loss function determines the predictive power and the regularization controls the complexity of the model. The target function is optimized using the gradient descent method. Given an objective function Obj(y,ŷ) to be optimized, the gradient descent is calculated in each iteration

∂ᵢⱼObj(y,ŷ)

and ŷ is changed along the descending gradient so that the objective function Obj is minimized.

To create the regression trees, internal nodes are divided based on a feature of the dataset. The resulting edges define the value range that allows the datasets to be divided. Leaves within the regression trees are weighted, wherein the weight corresponds to the predicted value. The number of iterations indicates how often the process of bagging and boosting is repeated. The XGBoost algorithm provides a very extensive list of hyperparameters, which contribute significantly to the formation of a good model.

Irrespective of the model used, correlations can be used to evaluate and represent linear relationships between two variables. The Pearson correlation coefficient r (or r²) provides a common measure for evaluating this relationship. It is dimensionless and is calculated according to:

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$

and varies within a range of −1≤r≤+1. The counter describes the sum of the deviation products of the two variables x and y to the mean, which corresponds to the empirical covariance s_{xy}. The denominator is the root of the product of the individual empirical standard deviations sₓ and s_{y}. The mean values of the quantity to be correlated are described as x̅ and y̅. The linear relationship according to Fahrmeir [23] can be interpreted with:
r<0.5: weak linear relationship
0.5≤r<0.8: medium linear relationship
0.8≤r: strong linear relationship

In the correlation analysis, it should be noted that only linear relationships can be shown here. The Bravais-Pearson correlation coefficient is therefore not suitable for describing non-linear relationships. This can mean that despite a correlation coefficient 0.0≤r≤0.2, there is a strong non-linear dependency of the variables.

Through mutual information, non-linear dependencies of two random variables can be determined. It is used in information theory [24]. With the help of the probabilities, the information content of a random variable compared to a second random variable is described. The basic formal relationship is:

$$I ( X ; Y ) = {\int }_{X} {\int }_{Y} {f}_{X , Y} log \frac{{f}_{X , Y} ( x , y )}{{f}_{X} ( x ) {f}_{Y} ( y )} .$$

This approach was accordingly expanded by Kraskov et al. and Ross et al. so that it could be used for the selection of suitable, continuous variables [25] [26].

Appropriate metrics must be used to compare different models. With the help of these, it is possible to make a statement about the accuracy with which a model can describe a target variable.

The coefficient of determination R² indicates which proportion of the variance of the target variable y can be described by the model. The coefficient of determination can be calculated according to:

$${R}^{2} ( y , \hat{y} ) = 1 - \frac{{\sum }_{i = 0}^{{n}_{Samples} - 1} {( {y}_{i} - {\hat{y}}_{i} )}^{2}}{{\sum }_{i = 0}^{{n}_{Samples} - 1} {( {y}_{i} - {\overline{y}}_{i} )}^{2}}$$

wherein ŷ is the estimated target variable of the i-th example and yᵢ is the associated true value. y̅ is the mean. The coefficient of determination can take values between 0 and 1. The closer the coefficient of determination is to 1, the better the model is able to fit the target variable.

The root mean squared error (RMSE) is another statistical measure that can be used to determine the model quality. Here the root of the mean, squared distance of the actual to the estimated value is calculated:

$${RMSE}_{h} = \sqrt{\frac{{\sum }_{i = 1}^{n} {( {y}_{i} - \hat{y} )}^{2}}{n}}$$

By squaring the error and then forming the root, the RMSE can be interpreted as the standard deviation of the variable to be estimated. Where n is the number of observations and ŷ is the estimated value of a target variable y. The representation of the error by the RMSE is an absolute error value that delivers values of different sizes depending on the target parameter examined. Therefore, it makes sense to relate the RMSE to the mean:

$$RMSE = \frac{{RMSE}_{h}}{\overline{y}}$$

Thus, the RMSE can be calculated relative to the mean true value y̅. This allows a better assessment of the error for target variables of different sizes.

### Methods

With the method according to the present invention, it is possible to determine the cell growth, i.e. the timeline of the cell density, and the timeline of certain metabolites, in particular glucose and lactate, in real time during a cultivation, especially on a small cultivation scale, from on-line process variables. With the method according to the current invention it is, thus, possible to provide real-time values for process variables that were previously not available in real time but only off-line. This represents an improvement over the conventional determination methods for cell growth and the timeline of certain metabolites, in particular glucose and lactate, insofar that the method according to the current invention does not require sampling from the cultivation medium.

In a preferred embodiment, the method according to the present invention is used to determine the cell density, the glucose concentration and the lactate concentration in a fed-batch cultivation of mammalian cells with a cultivation volume of 300 mL or less from on-line process variables, wherein the method is carried out without sampling, i.e. feedback control sampling.

The method according to the present invention allows cultivations to be carried out fully automatically, i.e. without sampling, on a small scale, i.e. with a cultivation volume of 300 mL or less, in which relevant process variables, such as cell density, cannot be determined on-line but only off-line.

The method of the present invention is particularly suitable for monitoring and controlling cultivations of mammalian cells on a small scale.

In the method according to the current invention, a method for determining the live cell density, glucose and lactate concentration as the target parameter in a CHO cell cultivation is provided, wherein the method employs a data-based soft sensor. Machine learning models are used to describe the different target variables.

The present invention is based, at least in part, on the finding that the selection of the process variables used for the model generation has a significant impact on the quality of the determined target process variables.

Furthermore, the present invention is based, at least in part, on the finding that the type of division, i.e. allocation, of the existing datasets into a training dataset and a test dataset influences the model quality.

Furthermore, the present invention is based, at least in part, on the finding that the type of antibody produced influences the choice of the optimal target parameter.

The method according to the current invention is described below using 155 exemplary datasets, which have been obtained from cultivations in the ambr250 system. This should not be understood as limiting the teaching according to the current invention or the method according to the current invention, but rather as an exemplary application of the teaching according to the current invention. Other datasets, which have been generated with the same or a different cultivation system, can equally well be used for and in the method according to the invention.

The 155 datasets were analyzed and examined for suitable features. Corresponding interpolation strategies were used to map the target parameters in such a way that the selected models could provide values for all target parameters at discrete points in time. The models were assessed with regard to errors and model quality. The methods based on it allowed for the provision of a robust and precise model of the respective target variable/process variable.

The molecular formats of the antibodies produced in the cultivations in the datasets differed. An overview of the various projects and molecular formats as well as the respective number of cultivations is shown in Table 1 below.

TABLE 1
Data overview.

| | | classifi- | classifi- | culti- | |
|---|---|---|---|---|---|
| project | molecule format | cation 1 | cation 2 | vations | medium |
| 1 | One-armed | Complex | Complex | 24 | A |
| | antibody Fc- | antibody | | | |
| | fusion | format | | | |
| 2 | Bispecific 1 + 1 | Y-shaped | Standard | 12 | B |
| | IgG format | antibody | | | |
| 3 | Bispecific 2 + 1 | Complex | Complex | 47 | C |
| | format with C- | antibody | | | |
| | terminal Fab | format | | | |
| | fusion | | | | |
| 4 | Bispecific 2 + 1 | Y-shaped | Complex | 72 | D |
| | format with N- | antibody | | | |
| | terminal Fab | | | | |
| | fusion | | | | |

The data, i.e. the on-line parameter set, associated with the entire cultivation process, and the associated date and time stamp were used for each cultivation. The data density of the different process values varied with regard to the timeline. These deviations in the data density can be attributed to the fact that, due to the system, a new data point was only recorded for an on-line parameter if the measured value was changed by a delta that was specifically defined for each measured value. In order to make continuous process data available and to ensure that the runs can be compared with one another, the corresponding on-line parameters were interpolated for all missing time stamps.

It should be noted for the on-line process variables that too much smoothing of the data would lead to a loss of fluctuations in the measured values. However, this noise also represents any process-relevant changes that are taking place and are contained in the process values as information. It is therefore important not to oversmooth the process values and to make changes in the process course accessible even after interpolation.

The off-line data contains different numbers of analysis values depending on the number of samples (between 8 and 13) during a cultivation. Each dataset contains a date and time stamp for each data point and the associated analysis values of the off-line parameters.

The preprocessing by interpolation of the on-line and off-line data results in a dataset, which contains the same number of data points for all process variables, regardless of whether they were on-line or off-line process variables, at the same times. The analysis was based on the interpolated dataset. Such an interpolation is not necessary if data points are available at the same frequency and at the same times for all on-line and off-line process variables.

Due to the preprocessing of the available on-line and off-line data, the different time profiles of the individual process variables due to different measurement frequencies are standardized to a uniform time profile, i.e. a single timeline. Bad values caused by technical and process management are identified and deselected or corrected, as well as existing time gaps are closed so that all process variables in one dataset for a cultivation and all datasets for all cultivations with regard to the time and number of process variables are uniform.

So that the fluctuations of the measurement signals caused by switching on the control at the beginning or switching it off at the end of the cultivation do not falsify the model formation, the data that was collected in the first and the last 12 hours of the cultivation was not used. In the specific example, this means that a time range from day 0.5 to day 13.5 was used. This guarantees that a change in the process variables can only be attributed to processes in the cell culture. The interpolation of the on-line data was carried out for the entire dataset. FIG. 1 shows an example of the linear interpolation of the process value ‘AO.PV’

As can be seen in FIG. 1, the course of the on-line signal with linear interpolation is well described. At the beginning (<day 0.5) it can be seen how the measured value fluctuated when the control is started. The peaks (larger process value changes within a short time) can also be mapped well with this type of interpolation.

For the off-line data, the obtained analysis values (VCD, VCV, glucose, lactate) were fitted with three different interpolations. FIG. 2 shows an example of the interpolation of the VCD with the different fit methods.

The respective coefficient of determination, R², was calculated to evaluate the individual interpolations for the VCD. The univariate spline achieved the highest R² value here, but tended towards significant overfitting. Accordingly, the univariate spline describes almost every measured value exactly, but does not depict a typical growth curve of a biological system. On the other hand, there are smaller differences between the Peleg fit and the polynomial fit. However, the Peleg fit can describe the different growth phases of biological systems much better and is therefore used for interpolation of the target variable for VCD [27].

The interpolation of the lactate and glucose profiles showed that the univariate spline maps the off-line data with better R² and describes the profile in the case of lactate much better. Since the polynomial fit interpolates negative values for lactate from day 10, the interpolations of the univariate spline were defined as target vectors y for lactate. For glucose, however, the polynomial fit (third degree) was used to describe the target variable (glucose: univariate spline (R²=0.999) and polynomial fit (R²=0.958); lactate: univariate spline (R²=0.999) and polynomial fit (R²=0.959)).

Furthermore, datasets, which contained too few off-line data points for preprocessing (three or less), were no longer used for the analysis. This was the case for two datasets. The entire interpolated and adjusted dataset therefore contained 153 cultivations.

Since the interpolated datasets with the maximum resolution of five minutes contained a large number of data points, the analysis was carried out with a resolution of 1/10 days only to reduce the computational effort. The JMP® program can be used for this.

FIG. 3 shows for the datasets from Project 2 (12 cultivations). As can be seen the different interpolation methods (Peleg fit, univariate spline and polynomial fit) have only a very small effect on the strength of the correlation.

In the scatter plots in FIG. 3, the on-line parameters are shown as a feature (lines). The columns represent the different interpolations of the VCD. The ellipses of the scatter plots always contain 95% of the data. The closer the ellipses are together, the stronger the linear relationship between the variables. The calculated Bravais-Pearson correlation coefficients are shown in Table 2 below.

TABLE 2
Numerical values of the Pearson correlation coefficients for the
sample dataset from Project B corresponding to that of FIG. 3.

| | VCD | VCD | VCD |
|---|---|---|---|
| | [ES cells/ml]_pelegfit | [ES cells/ml]_Spline | [ES cells/ml]_Polyfit |
| VCD [ES cells/ml]_pelegfit | 1.0000 | 0.9895 | 0.9870 |
| VCD [ES cells/ml]_Spline | 0.9895 | 1.0000 | 0.9903 |
| VCD [ES cells/ml]_Polyfit | 0.9870 | 0.9903 | 1.0000 |
| ACO.PV [%] | 0.6757 | 0.6724 | 0.6637 |
| AO.PV [%] | 0.8989 | 0.8953 | 0.8929 |
| CO2.PV [ml/min] | 0.4586 | 0.4579 | 0.4532 |
| DRZ.PV [rpm] | 0.6493 | 0.6511 | 0.6453 |
| FED2T.PV [ml] | 0.8254 | 0.8185 | 0.8212 |
| FED3T.PV [ml] | 0.8611 | 0.8559 | 0.8560 |
| GEW.PV [ml] | 0.8266 | 0.8196 | 0.8226 |
| LGE.PV [ml] | 0.9130 | 0.9002 | 0.9016 |
| N2.PV [ml/min] | −0.9528 | −0.9441 | −0.9443 |
| O2.PV [ml/min] | 0.9547 | 0.9490 | 0.9490 |
| PO.PV [%] | 0.0058 | 0.0253 | 0.0235 |
| pH.PV | 0.3357 | 0.3464 | 0.3319 |

Looking at the value of ‘O2.PV’ as an example, the calculated coefficients for the interpolations are very close to each other (0.9547; 0.9490; 0.9490).

The correlation analysis was accordingly carried out on the entire dataset. Table 3 below shows the Bravais-Pearson correlation coefficients determined in this way.

TABLE 3
Calculated Pearson correlation coefficients for the entire dataset
(153 cultivations), target variable VCD fitted for the Peleg fit.

| | On-line parameter | r |
|---|---|---|
| | Time | 0.4756 |
| | ACO.PV | 0.6188 |
| | ACOT.PV | 0.3878 |
| | AO.PV | 0.2530 |
| | CHT.PV | 0.2939 |
| | CO2.PV | 0.4367 |
| | CO2T.PV | 0.3680 |
| Correlation | DRZ.PV | 0.0109 |
| analysis | | |
| | FED2T.PV | 0.5085 |
| | FED3T.PV | 0.3864 |
| | GEW.PV | 0.6090 |
| | LGE.PV | 0.7503 |
| | N2.PV | −0.8313 |
| | O2.PV | 0.0189 |
| | OUR | 0.0631 |
| | PO.PV | −0.0570 |
| | PH.PV | 0.2292 |

Compared with the correlation analysis on a single ambr250 run (see the previous Table 3 and FIG. 3), the correlation analysis showed significantly weaker linear relationships across the entire dataset. Apart from the strength of the correlation, the analysis of the entire dataset also produced other on-line parameters as the best candidates. It was also found that the independent variables correlate with each other. The following Table 4 shows in part the correlation of the parameters ‘O2.PV’ and ‘N2.PV’ to the other independent variables.

TABLE 4
Correlation of the independent variables to each other,
shown using the example of O2.PV and N2.PV, which
had the highest correlation values in the previously
performed correlation analysis of Project B.

| | O2.PV | N2.PV |
|---|---|---|
| | [ml/min]_interp1d | [ml/min]_interp1d |
| O2.PV [ml/min]_interp1d | 1.0000 | −0.8386 |
| N2.PV [ml/min]_interp1d | −0.8386 | 1.0000 |
| ACO.PV [%]_interp1d | 0.6901 | −0.6296 |
| AO.PV [%]_interp1d | 0.8150 | −0.9349 |
| CO2.PV [ml/min]_interp1d | 0.5314 | −0.4853 |
| DRZ.PV [rpm]_interp1d | 0.4205 | −0.5093 |
| FED2T.PV [ml]_interp1d | 0.7016 | −0.6277 |
| FED3T.PV [ml]_interp1d | 0.4903 | −0.4828 |
| GEW.PV [ml]_interp1d | 0.7776 | −0.6975 |
| LGE.PV [ml]_interp1d | 0.7742 | −0.7511 |
| PO.PV [%]_interp1d | −0.0279 | 0.0338 |
| pH.PV_interp1d | 0.4056 | −0.3739 |

When independent variables correlate with one another, one refers to multicolinearity. As shown here using the example of ‘O2.PV’, there is a clear linear relationship between the two best correlation coefficients for ‘N2.PV’ and ‘O2.PV’ from FIG. 3 and the remaining independent parameters.

FIG. 4 shows the calculated information content (mutual information) for all the features for the target variable VCD for the entire dataset. FIG. 4 shows that some of the available features have a high level of information about the VCD target variable. With regard to the VCD, the mutual information could thus have the highest index for ‘Time’, CHT.PV′, ‘ACOT.PV’, ‘FED2T.PV’, ‘GEW.PV’, ‘CO2T.PV’, ‘ACO.PV’, ‘AO.PV’, ‘O2.PV’, ‘N2.PV’ and ‘LGE.PV’.

Based on the calculation of the information content and the results of the correlation analysis, the best ten process variables (CHT.PV, ACOT.PV, FED2T.PV, GEW.PV, CO2T.PV, ACO.PV, AO.PV, LGE.PV, O2.PV and N2.PV) are selected and a corresponding feature matrix X is created. The matrix contains the interpolated data of the available dataset. A resolution of five minutes for the feature (f₁ . . . f₁₀) and the duration of the cultivation in hours were selected as an additional column in the matrix:

$$X = \begin{matrix} \\ Run 1 \\ ⋮ \\ Run 1 \\ ⋮ \\ Run 153 \\ ⋮ \\ Run 153\end{matrix} \begin{matrix}\begin{matrix}Time in hours & {f}_{1} & {f}_{2} & … & {f}_{10}\end{matrix} \\ ( \begin{matrix}0.5 & Run {1}_{{f}_{1}} & Run {1}_{{f}_{2}} & … & Run {1}_{{f}_{10}} \\ ⋮ & ⋮ & ⋮ & ⋮ & ⋮ \\ 13.5 & Run {1}_{{f}_{1}} & Run {1}_{{f}_{2}} & … & Run {1}_{{f}_{10}} \\ ⋮ & ⋮ & ⋮ & ⋮ & ⋮ \\ 0.5 & Run {153}_{{f}_{1}} & Run {153}_{{f}_{2}} & … & Run {153}_{{f}_{10}} \\ ⋮ & ⋮ & ⋮ & ⋮ & ⋮ \\ 13.5 & Run {153}_{{f}_{1}} & Run {153}_{{f}_{2}} & … & Run {153}_{{f}_{10}}\end{matrix} )\end{matrix}$$

The division into training and test datasets was done in such a way that these were exclusively datasets from cultivations from Project 2. The target variable ‘VCD’ was divided according to the distribution of the feature matrix.

To check the quality of the models obtained, the relative frequency density of the errors on the entire test dataset was calculated. The histograms of the prediction on the entire test dataset of the model determined using the MLPRegressor (a), random forest (b) and XGBoost (c) for the target variable VCD showed on the X-axis the error of the estimated VCD values compared to the predicted values, and on the Y-axis the relative frequency of the errors. All three distributions show a left skew tendency, which indicates an underestimation of the VCD. Furthermore, examination of all the histograms shows that the estimates of all three models yield comparable results. The XGBoost shows the most homogeneous distribution of the calculated errors, although here also an overestimation of the target variable can be seen.

For each model, the RMSE and R² were calculated based on the entire test dataset. Both values relate to the Peleg fit of the target variable VCD. The results of the three models are summarized in Table 5 below.

TABLE 5
Results of the estimation on the VCD for
MLPRegressor, random forest and XGBoost.

| Model | RMSE | R² |
|---|---|---|
| MLPRegressor | 0.38 | 0.58 |
| Random forest | 0.33 | 0.67 |
| XGBoost | 0.34 | 0.66 |

All models achieved comparable results with regard to the RMSE and the coefficient of determination.

If one examines some specific datasets that were determined with random forest (best model), it can be seen that it is not possible to accurately map the Peleg fit of the VCD over the entire cultivation period (see FIG. 5). From day 5, the model in the upper portion of the figure is unable to correctly depict the relationship of the data to the VCD. The lower portion of the figure shows opposite behavior. The model estimates a too high VCD right from the start and therefore cannot achieve a sufficiently precise description of the VCD.

It has now surprisingly been found that the exchange of features in the feature matrix with a high information content for those with a significantly lower information content but with a still determinable information content can significantly increase the quality of the prediction.

It has been found that the extension of the matrix by the features ‘CO2.PV’, ‘FED3T.PV’, ‘OUR’ and ‘PH.PV’ and the deletion of the redundant feature ‘O2.PV’ (gassing with N₂ and O₂) leads to an improved quality of the prediction.

The improved feature matrix contains the following 14 features: ‘Time’, ‘ACO.PV’, ‘ACOT.PV’, ‘AO.PV’, ‘CHT.PV’, ‘CO2.PV’, ‘CO2T.PV’, ‘FED2T.PV’, ‘FED3T.PV’, ‘GEW.PV’, ‘PH.PV’, ‘N2.PV’, ‘IGE.PV’ and ‘OUR.PV’.

Furthermore, it has been found that the selection or the division into training and test datasets has an influence on the quality of the prediction.

When comparing the training and test datasets already selected with regard to the target variable, it was found that the distribution of the VCD for the training dataset consisting of cultivations from Project 2 had a mean value μ_{Train}=84.60, with a standard deviation of σ_{Train}=48.62, while the test dataset had a mean value of μ_{Test}=64.22, with a standard deviation of σ_{Test}=38.02.

It has been found that it is disadvantageous to take training datasets from only one project if the prediction is to be made for cells that express structurally different proteins. It has been found that it is advantageous to randomly distribute the training datasets over the entirety of the existing datasets.

In the present example, in order to distribute the datasets more homogeneously, 30 random numbers between 0 and 152 (since there were 153 datasets) were generated. The numbers stood for one cultivation run each. The random numbers were repeatedly generated until comparable mean values and standard deviations regarding the division between test and training dataset could be achieved with the trained model. The final division resulted in μ_{Train}=80.72 with σ_{Train}=47.11 and μ_{Test}=80.11 with σ_{Test}=48.70 and was used as the split ratio of the two datasets in the further course.

Thus, in one embodiment of the method according to the invention, the existing, preferably preprocessed, datasets are divided into a training dataset and a test dataset, wherein the training dataset is 70-80% of the total dataset (in this example 80% and, thus, 123 cultivation runs) and the test dataset contains 20-30% of the data of the entire dataset (in this example, 30 randomly selected cultivations of the entire dataset that were validated as described above were available for the validation of the models).

The models were then trained and tested with the extended feature matrix as well as with the new distribution of the datasets. The strategy for optimizing the hyperparameters as outlined above has been retained for this. From the corresponding histograms of the estimates for the VCD with the newly divided training and test datasets, it can be seen that the distribution of the errors for all three models have become significantly narrower, which can be attributed to a more precise estimate of the target parameter (FIG. 6).

All three models can achieve an error distribution that fluctuates more clearly around the true value (X-axis at 0) of the target variable. Here too, the histogram of the XGBoost shows the most homogeneous error distribution. The histogram of the random forest shows minor errors over the entire area. If the two histograms (a) and (c) are compared with each other, the XGBoost more often estimates the exact target value than the MLPRegressor. However, due to the width of the distribution of the MLPRegressor with a low degree of error, one can infer about the same accuracy for both models.

TABLE 6
Results of the estimation on the VCD for MLPRegressor, random forest
and XGBoost with new distribution of the test and training data.

| Model | RMSE | R² |
|---|---|---|
| MLPRegressor | 0.23 | 0.86 |
| Random forest | 0.20 | 0.89 |
| XGBoost | 0.22 | 0.85 |

All three models are able to achieve closely related results. FIG. 7 shows an exemplary estimate of the best model using individual cultivations.

Nearly ideal estimates of the target variable based on the Peleg fit of the raw data are thus achieved. Looking at the entire test dataset, all models are able to achieve good results with regard to the R² and RMSE with the split ratio of the datasets as described above.

The glucose values fitted by a third degree polynomial fit were used as the target parameter for the estimation of the glucose concentration. The feature matrix used for the training contained the same features as for the VCD. The same division into training and test datasets was also used.

Just as for the VCD, the histograms show comparable results in terms of errors. Here, too, the XGBoost can most often achieve a small error between the actual and estimated values. The random forest histogram also shows minor errors between the interpolated value of the target variable and the estimate, which are distributed homogeneously around the actual value of the glucose. The MLPRegressor shows the largest errors compared to the other two histograms.

TABLE 7
Results of the glucose estimate for MLPRegressor,
random forest and XGBoost.

| Model | RMSE | R² |
|---|---|---|
| MLPRegressor | 0.13 | 0.94 |
| Random forest | 0.12 | 0.95 |
| XGBoost | 0.14 | 0.93 |

FIG. 9 shows two exemplary cultivations obtained with random forest. The target variable was aptly described with a coefficient of determination of 0.93.

For the estimation of the lactate concentration, the values of the lactate fitted by the univariate spline method were used as the target parameter. The feature matrix used for the training contained the same features as for the VCD and glucose. The same division into training and test datasets was also used. The histograms show different results regarding the errors (FIG. 11).

If the histogram of the MLPRegressor is considered, an estimate with small errors is not possible as often as for the other two models. The random forest and XGBoost, on the other hand, have very narrow distributions. It seems that for some estimates of the target variable, very good predictions can be made with few errors, but these quickly lead to more significant errors within the entire test dataset. The neural network has the most homogeneous error distribution here.

Table 8 below shows the results of the lactate evaluation for RMSE and R² for all models.

TABLE 8
Lactate estimation results for MLPRegressor,
random forest and XGBoost.

| Model | RMSE | R² |
|---|---|---|
| MLPRegressor | 0.18 | 0.95 |
| Random forest | 0.35 | 0.81 |
| XGBoost | 0.27 | 0.88 |

FIG. 12 shows the predicted values of the XGBoost for lactate for an exemplary cultivation from the test dataset. A nearly ideal description of the fitted lactate course can be seen in the upper partial image; in the lower part, the course could be described with an R² of 0.98.

For the validation, first there was a study to determine which of the models is most efficiently able to describe the interrelationships of the feature on the test dataset. For this purpose, the models were initially only provided with ten datasets for learning. As the process progressed, the number increased by ten datasets each. This resulted in twelve training processes in which the models received between 10 and 120 datasets. After each training session, the target variable was estimated based on the test dataset. The respective RMSE was calculated. The test dataset also consisted of the 30 randomly validated selected datasets, as described above. VCD was chosen as the target variable. This resulted in the learning behavior described in FIG. 13.

As FIG. 13 shows, the random forest and XGBoost are both able to achieve a smaller error in the prediction of the test dataset with a small number of datasets than the neural network. However, this effect seems to diminish as the number of training datasets increases, so that comparable errors compared to the other two models can be achieved from around 80 datasets onward. With the maximum number of 120 datasets, the random forest achieves the lowest RMSE. However, the errors of all models are in a very narrow range.

A detailed evaluation of the estimate of the models regarding the prediction of the VCD for the 30 cultivations of the test dataset was carried out. Despite the good results across the entire dataset (histograms, coefficient of determination, RMSE), it was found that some predictions still showed a significantly larger deviation. FIG. 14 shows a cultivation run in which the estimated course of the VCD is clearly above the real distribution.

The insufficient accuracy of the estimate was increasingly observed in cultivations from Projects 1 and 3. In cultivations from both projects, the cultivated cells produced a complex molecular format.

It was found that the VCD in IgG-based formats, which have the characteristic Y-shape of a natural IgG antibody or largely retain it (Projects 2 and 4), is higher on average than in cells that have a complex molecular format as the target product (projects 1 and 3) and that the calculated cell diameter had a higher value for projects with complex molecular formats.

FIG. 15 shows the average cell diameter of the projects grouped by Y-shaped IgG (IgG, Projects 2 and 4) and complex IgG (complex, Projects 1 and 3) for each sample, as well as the standard deviation in the form of a box plot diagram. The figure shows that the green box plots (complex protein formats; left at each time point) lie above the blue box plots (Y-shaped IgG antibodies; right at each time point). At the beginning of the cultivation period, both molecular formats are still relatively close together. The cells with complex molecular formats as the target product only become significantly larger as the cultivation period progresses. In contrast, it can be seen that the cells with standard antibodies grow larger until day 7, but then no further increase in the diameter of the cells can be found.

It was found that the relationship between a higher VCD and a smaller cell diameter for IgG formats, as well as the smaller VCD and larger cells in complex protein formats, causes the inaccurate prediction of VCD.

It has also been found that the viable cell volume (VCV) represents a more suitable target variable than the VCD, not only for those cultivations in which a complex antibody format is produced, but also for cultivations in which a Y-shaped IgG antibody is produced.

The VCV is calculated using the following formula:

$$VCV = VCD \cdot \frac{4}{3} \cdot π \cdot {( \frac{D}{2} )}^{3} .$$

The VCV is therefore a better approximation for describing the living biomass in a cultivation than the VCD.

Since the calculated values of the VCV, like all other off-line parameters, only contained times of the sampling, the new target parameter was fitted with a third-degree polynomial fit. The models were then trained and evaluated for the new target size, as already described for the other target parameters above.

The RMSE and the coefficient of determination were used to evaluate the individual models. In summary, the best models with 14 features achieved the following results:

TABLE 9
Comparison of the RMSE and coefficient of determination
of the best models against the target variable VCD

| Model | RMSE | R² |
|---|---|---|
| MLPRegressor | 0.23 | 0.86 |
| Random forest | 0.20 | 0.89 |
| XGBoost | 0.22 | 0.85 |

For the target variable VCV, the calculated errors and coefficient of determination of the individual models are summarized in the following Table 10:

TABLE 10
Comparison of the RMSE and coefficient of determination
of the best models against the target variable VCV

| Model | RMSE | R² |
|---|---|---|
| MLPRegressor | 0.14 | 0.94 |
| Random forest | 0.11 | 0.97 |
| XGBoost | 0.12 | 0.96 |

By using the target variable VCV instead of VCD, all models were able to achieve coefficients of determination above 0.9. The improvement in the models can be seen in both, a lower RMSE and higher R² values.

In order to demonstrate the improved results in the comparison between the live cell density and the cell volume, scatter plots were obtained which represent both the estimate for the entire training set and the test dataset. Random forest estimates the best results for VCD and VCV. The two scatter plots are shown in FIG. 16.

If the two scatter plots are compared with each other, it can be seen that the prediction of the VCV is closer to the ideal estimate and has a significantly smaller spread of the test and training datasets than the prediction of the VCD. If only the training data (blue dots) are considered, the models learn the relationships of the feature better in relation to the cell volume than to the live cell density. These features therefore allow a more precise estimate of the cell volume for the entire test dataset for all trained models.

The extent to which the division of the antibodies into different groups and the use of only limited datasets with regard to the training of the method influence the quality was investigated as follows.

If all four projects are considered separately with regard to the course of the target parameter VCV, the box plot shown in FIG. 17 is obtained. As can be seen, the VCV in Project 4 behaves between those of Projects 1 and 3 on one side and Project 2 on the other side. This means that the datasets from Projects 1, 3, and 4 can also be classified as complex IgG antibody formats (classification 2). Therefore, the calculations were repeated with this classification. Various combinations of training and test datasets were also tested. The results are shown in Table 11 and FIGS. 18 and 19.

TABLE 11
RMSE for different combinations of training and test datasets.

| Training | | | | | |
|---|---|---|---|---|---|
| with data | Prediction | | Target | | |
| from . . . | for . . . | | param- | RMSE | RMSE |
| antibodies | antibodies | Model | eter | (train) | (prediction) |
| Standard | Complex | MLPRegressor | VCD | 0.2242 | 0.9111 |
| Standard | Complex | Random forest | VCD | 0.0733 | 0.5647 |
| Standard | Complex | XGBoost | VCD | 0.145 | 0.4718 |
| Standard | Complex | MLPRegressor | VCV | 0.4825 | 0.8456 |
| Standard | Complex | Random forest | VCV | 0.0551 | 0.3413 |
| Complex | Standard | MLPRegressor | VCD | 0.1932 | 0.4752 |
| Complex | Standard | Random forest | VCD | 0.0799 | 0.3343 |
| Complex | Standard | XGBoost | VCD | 0.0087 | 0.3545 |
| Complex | Standard | MLPRegressor | VCV | 0.1408 | 0.2772 |
| Complex | Standard | Random forest | VCV | 0.0411 | 0.2296 |
| Complex | Standard | XGBoost | VCV | 0.0237 | 0.2455 |
| Complex | Complex | MLPRegressor | VCD | 0.1933 | 0.2283 |
| and | and | | | | |
| standard | standard | | | | |
| Complex | Complex | Random forest | VCD | 0.0959 | 0.2004 |
| and | and | | | | |
| standard | standard | | | | |
| Complex | Complex | XGBoost | VCD | 0.0092 | 0.2172 |
| and | and | | | | |
| standard | standard | | | | |
| Complex | Complex | MLPRegressor | VCV | 0.1548 | 0.1443 |
| and | and | | | | |
| standard | standard | | | | |
| Complex | Complex | Random forest | VCV | 0.0705 | 0.1079 |
| and | and | | | | |
| standard | standard | | | | |
| Complex | Complex | XGBoost | VCV | 0.0592 | 0.1191 |
| and | and | | | | |
| standard | standard | | | | |

The different combinations have shown that the prediction using the random forest method has achieved the best results, i.e. the lowest RMSE.

The RMSE showed a significant improvement (reduction) in all combinations of the training or test datasets when the VCV was used as the target parameter compared to the VCD.

The different combinations of training and test datasets showed that the selection of the datasets depending on the molecular format influence the RMSE of the target parameter. In case of model training with datasets of a standard format and the estimation of the VCD or VCV of complex formats, this combination achieves the highest RMSE. Training with datasets of the complex molecular formats and prediction of the VCD or VCV led to a smaller RMSE. The smallest RMSE could be achieved if a mixed dataset was used for standard Y-IgG and complex molecular formats.

Furthermore, the models were evaluated with regard to the estimate of the training and test dataset in order to check whether the models already trained had been overfitted. The trained models for the target variable VCV were estimated on the test and training dataset. The estimated values were evaluated according to the RMSE and the difference between the test and training dataset was then shown in the form of a bar chart (FIG. 20).

FIG. 20 shows that the MLPRegressor achieves a lower error for the test dataset than for the training dataset. Therefore, the calculated difference is negative. The random forest and XGBoost have larger errors on the test dataset, which makes the difference shown here positive. Both models based on decision trees therefore tend to overfit.

### THE PRIOR ART

The prior art uses parameters such as Glucose, Lactate, Ammonia, VCD etc (all of which are off-line parameters) as input variables for a random forest regression analysis to explain the dynamic behavior of intracellular activities but not for the prediction or modelling of off-line parameters.

In contrast to the prior art, in the current invention the parameters used for the machine learning models are exclusive online-parameters (which are used to control fermentation conditions).

The current invention, thus, makes use of typical online measurement parameters, which are generated throughout the cultivation and a statistical model to estimate parameters like VCV, glucose, etc., without the need for an additional sensor or sampling.

### SUMMARY AND OUTLOOK

By interpolating existing on-line and off-line cultivation datasets, a standardized and uniform dataset could be obtained, which was used for a model generation to predict target parameters that are only available off-line.

For the off-line data, which was considered a target variable in the further course, it was essential to find an interpolation that can representatively describe the course for the respective target parameter. Since the live cell density relates to the growth course of a biological system, conventional interpolations such as the polynomial fit or the univariate spline fit can often only describe this target parameter with insufficient accuracy. An incorrect extrapolation would lead to a falsified description of the target variables. Although the selected interpolations yielded comparable results with regard to R², the selected interpolation according to M. Peleg [27] is best able to describe growth processes of cell culture processes. The background of the interpolation strategy lies in the combination of a continuous logistic equation for the description of the growth of the cells and the mirrored logistic equation for the description of the death behavior (Fermi's equation).

The result of the correlation analysis is only marginally unaffected by the choice of the interpolation strategy.

The accuracy of the estimate for the VCD target variable could be increased by an adapted split ratio of the datasets into training datasets and test datasets. For this purpose, the validation dataset was selected with regard to the distribution of the target variables so that the mean values and standard deviations were as small as possible from one another. The goal was not to artificially generate a better dataset for prediction. Rather, it was assumed that the test dataset previously generated could not be used to describe the overall dataset with sufficient accuracy. Reference is hereby made to cross-validation as a corresponding method.

The calculation of the cell volume and the related relation to the size of the cells could describe a better approximation of the biomass than the VCD, which, thus, resulted in VCV as a new target parameter.

The calculated cell volume as an approximation of the description of the biomass provided a higher information content about the process characteristics than the previously employed live cell density of the culture determined by analysis of the samples. The average volume of the cell culture can be concluded from the measured average diameter of the cells. It could be shown that the size of the cells, especially those with complex target molecules as a product, increases continuously with increasing cultivation time. However, the live cell density cannot map this relationship. In the end, the metabolic activity of the cultured cells can be better described by the viable cell volume than by the live viable cell density.

For real-time determination of the target parameters, the estimate should be made at a defined interval, for example ten minutes. For CHO cells, this interval is an acceptable resolution because they have a doubling time of approximately 24 hours.

The following Examples and Figures serve only to illustrate the invention. The scope of protection is defined by the pending claims. However, modifications to the disclosed embodiments can be made without departing from the principle according to the invention.

### DESCRIPTION OF THE FIGURES

FIG. 1: Linear interpolated measured value using the example of ACO.PV. Interpolation range from day 0.5 to day 13.5.

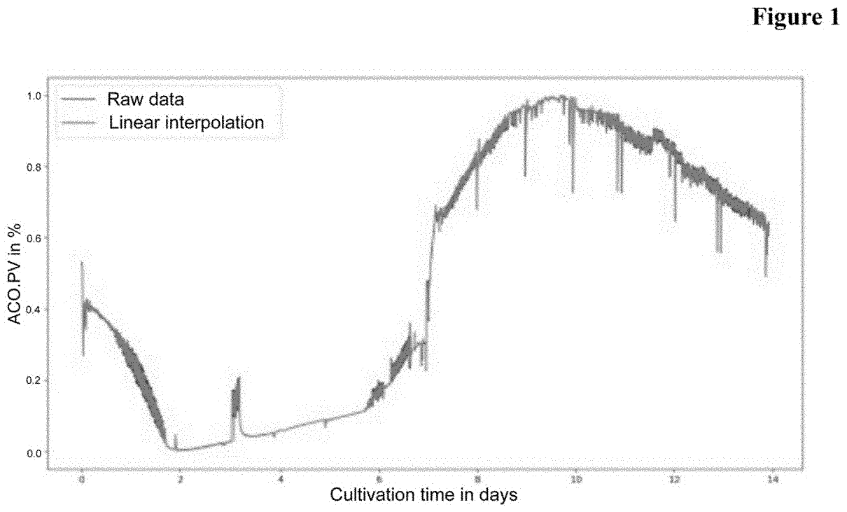

FIG. 2: Interpolated measurement curve of the live cell density of an exemplary cultivation. Interpolation and coefficient of determination: Peleg fit (R2=0.957), univariate spline (R2=0.998) and third degree polyfit (R2=0.864).

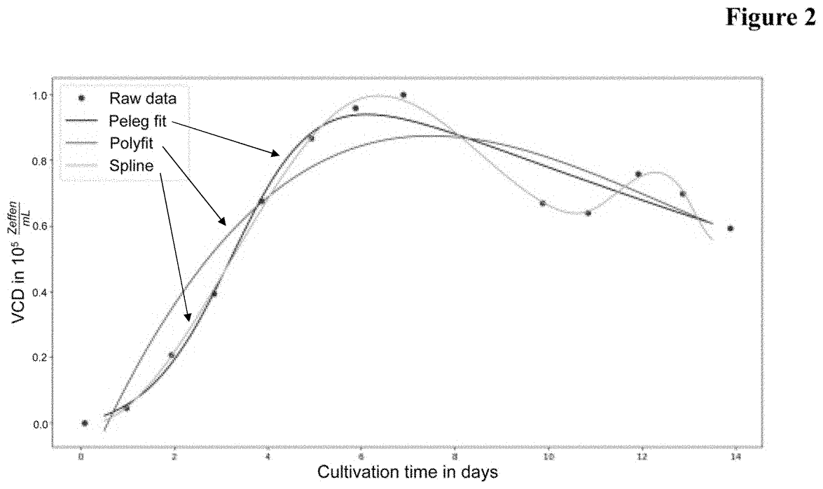

FIG. 3: Exemplary correlation analysis of the dataset of an ambr250 run from Project 2. Comparison of the correlation coefficients on the different interpolation strategies. The diagram shows the scatter plots of the individual on-line parameters for the VCD.

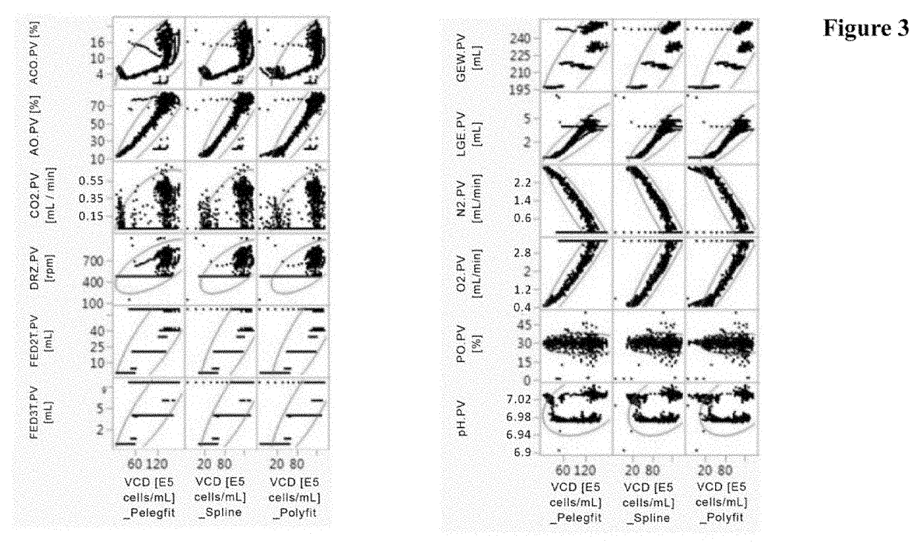

FIG. 4: Information content calculated according to the mutual information for the target variable VCD on the entire dataset.

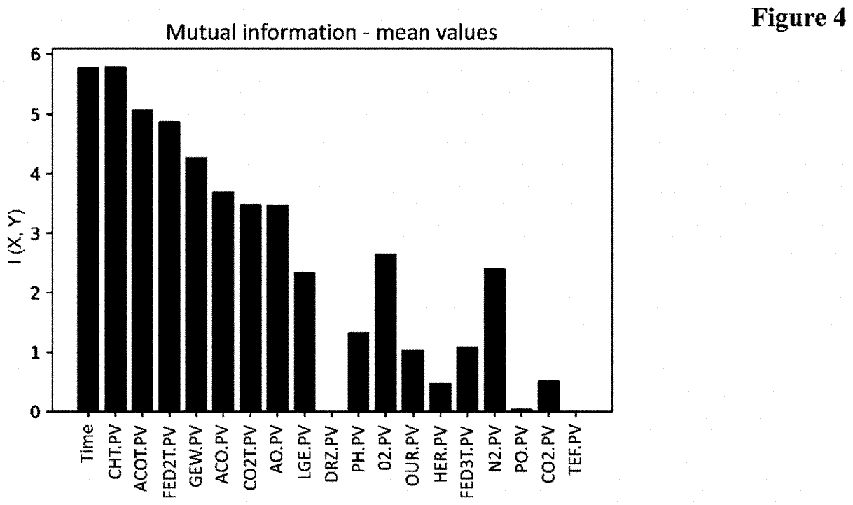

FIG. 5: Estimation of the random forest VCD for two separate runs. An estimate with an R2 of 0.20317 could be achieved in the upper portion of the figure. In the lower portion of the figure, an estimate of R2 of 0.54896 could be achieved.

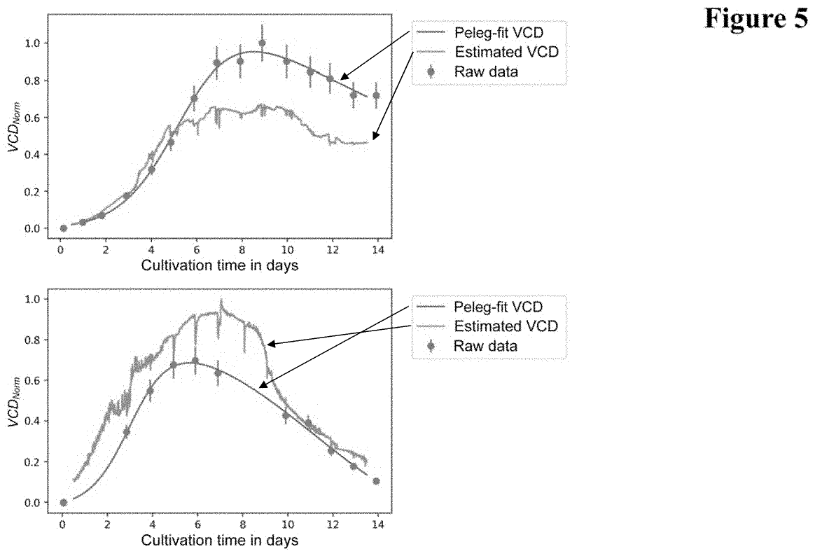

FIG. 6: Histogram of the prediction of the newly created test dataset of the models MLPRegressor (a), random forest (b) and XGBoost (c) for the target variable ‘VCD’. The error for the fitted VCD values for the predicted values is shown on the X-axis. The Y-axis indicates the relative frequency of the errors.

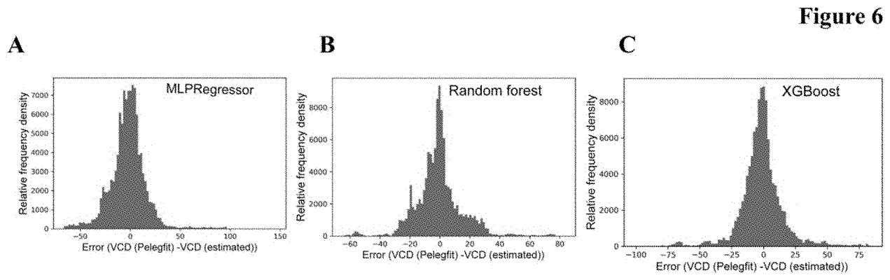

FIG. 7: Estimation of the VCD of the random forest for two exemplary runs of the test dataset. In the upper portion of the figure, an estimate of R2 of 0.98944 was achieved. An estimate of R2 of 0.99837 could be achieved in the lower portion of the figure.

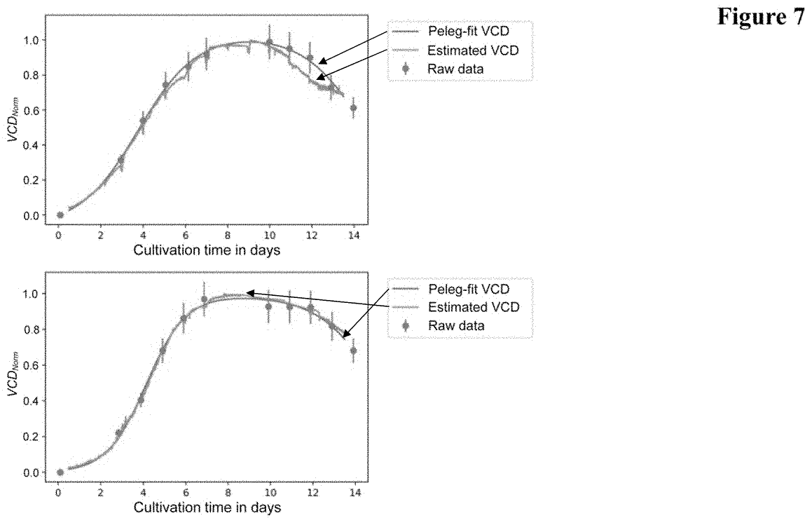

FIG. 8: Information content calculated for the entire dataset according to mutual information for the target variable glucose.

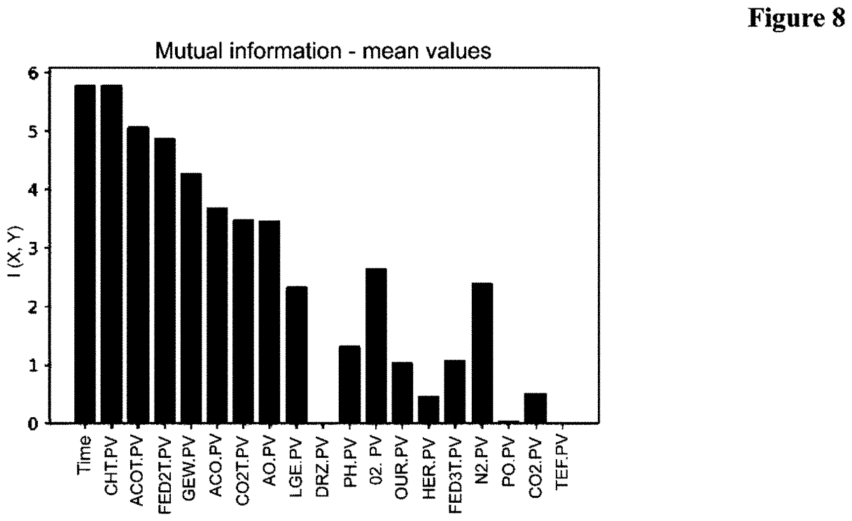

FIG. 9: Estimation of the glucose from the random forest for two exemplary runs of the test dataset. In the upper portion of the figure, an estimate of R2 of 0.99 could be achieved. In the lower portion of the figure, an estimate of R2 of 0.97 could be achieved.

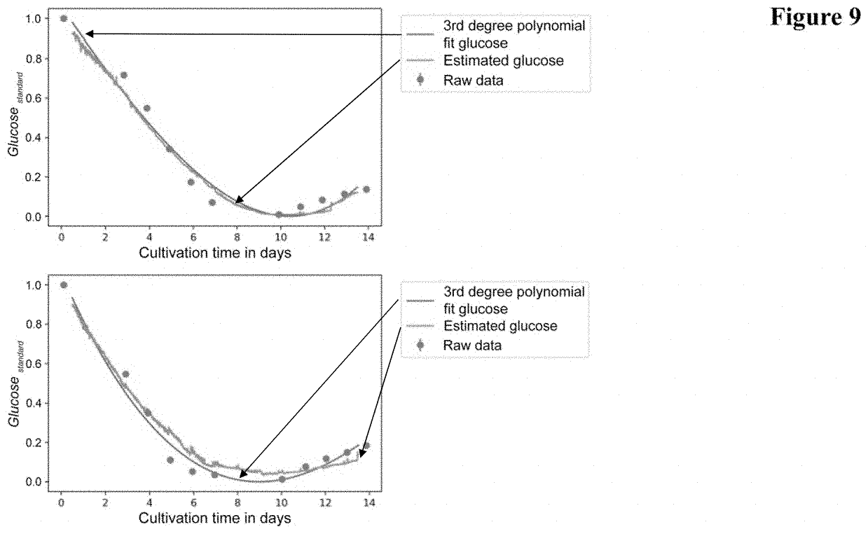

FIG. 10: Information content calculated for the entire dataset according to mutual information for the target variable lactate.

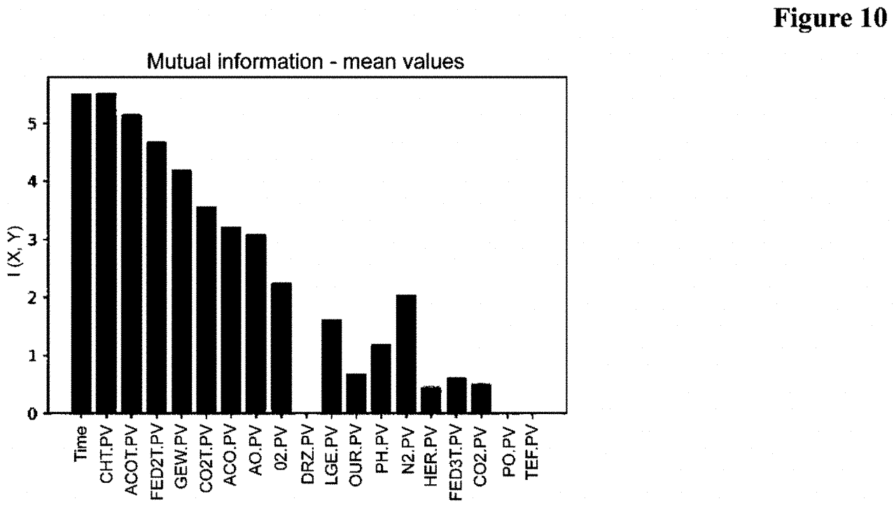

FIG. 11: Histograms of the prediction for the test dataset of the MLPRegressor (a), random forest (b) and XGBoost (c) for the target variable lactate. The error for the lactate values added to the predicted values is shown on the X-axis. The Y-axis indicates the relative frequency of the errors.

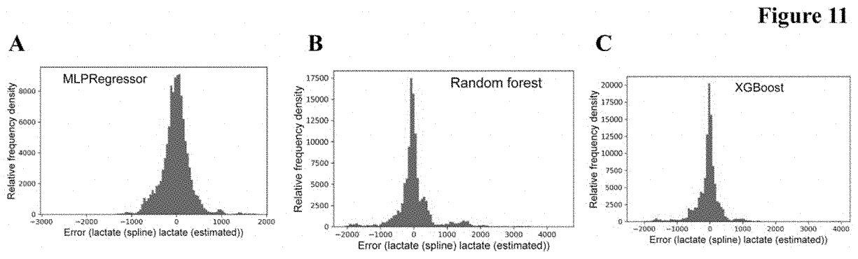

FIG. 12: Estimation of lactate by the XGBoost for two exemplary runs of the test dataset. In the upper portion of the figure, an estimate of R2 of 0.99 could be achieved. In the lower portion of the figure, an estimate of R2 of 0.98 could be achieved.

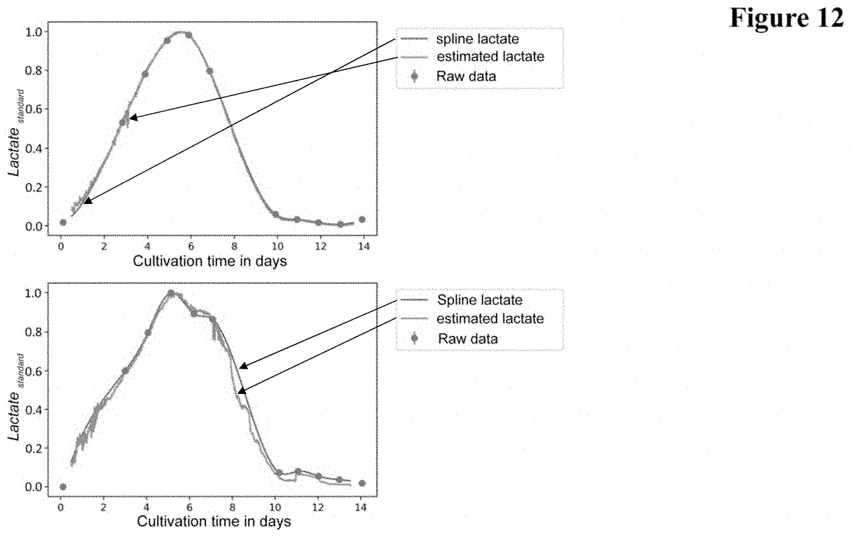

FIG. 13: Calculated RMSE for MLPRegressor, random forest and XGBoost with a different number of training datasets.

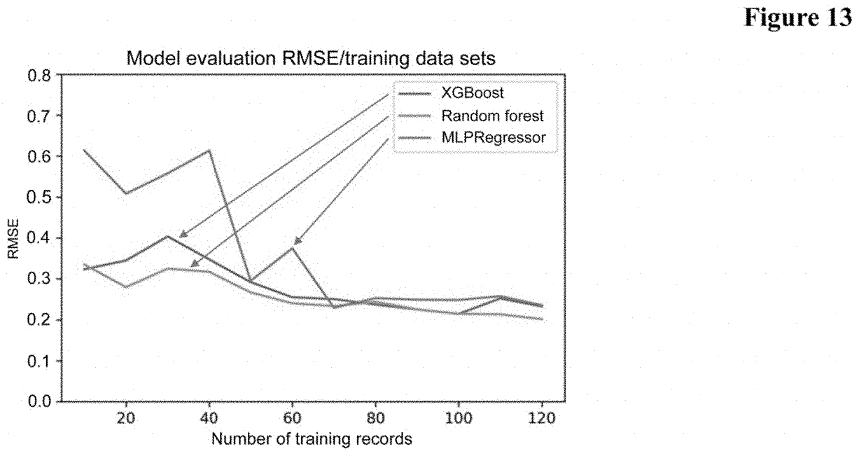

FIG. 14: Estimation of the random forest VCD for a single cultivation. The Peleg fit of the VCD is shown in blue, the estimated values for the VCD in orange.

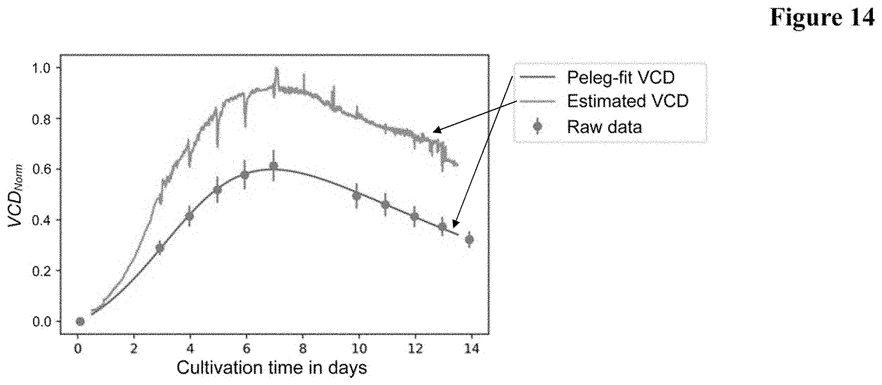

FIG. 15: Representations of the average diameter for each sampling over the entire cultivation period. Projects 1 and 3 have a complex molecular format (shown here in blue, left) as a product. Projects 2 and 4 have a Y-shaped Ig-G format (shown here in green, right) as the target product. Box plots contain the mean; the units were shown standardized.

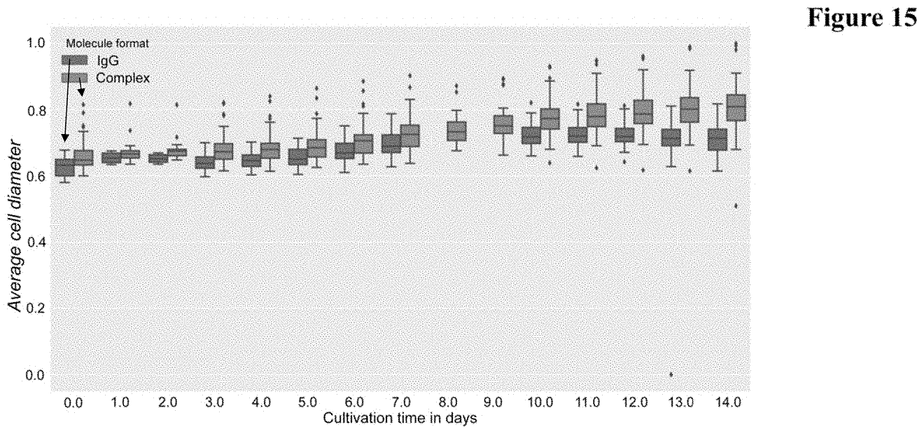

FIG. 16: Left portion of the figure: Estimation of the random forest on the VCD. In red, the estimated values for the test dataset against the true values. In blue, the estimated values for the training dataset against the true values. An ideal estimate for the test and training datasets is shown in black. Right portion of the figure: Estimation of the random forest on the VCV. In red, the estimated values for the test dataset against the true values. In blue, the estimated values for the training dataset against the true values. An ideal estimate for the test and training datasets is shown in black.

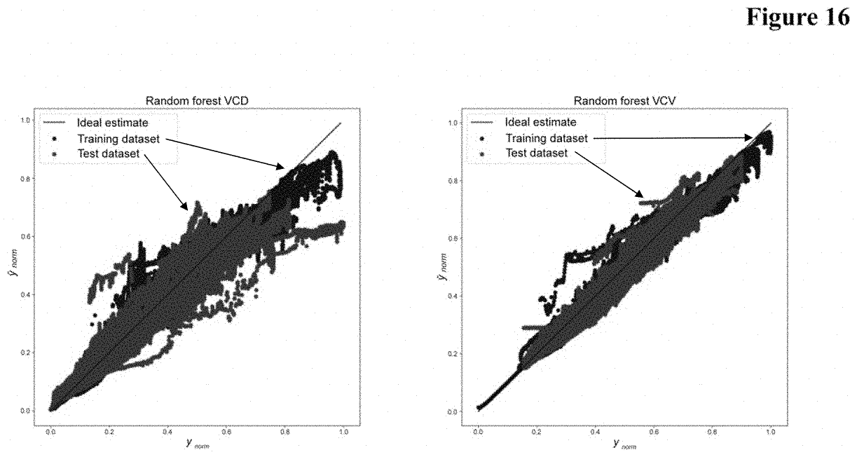

FIG. 17: Representation of the average diameter for each sample over the entire cultivation period for each project. Project 1=purple, Project 2=red, Project 3=green and Project 4=blue. Box plots contain the mean.

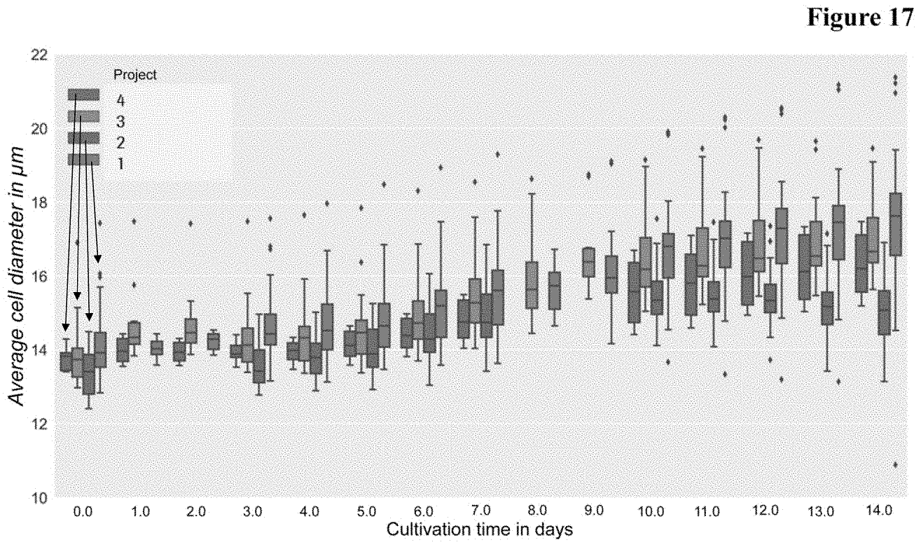

FIG. 18: Compare VCD/VCV using the random forest model (best model).

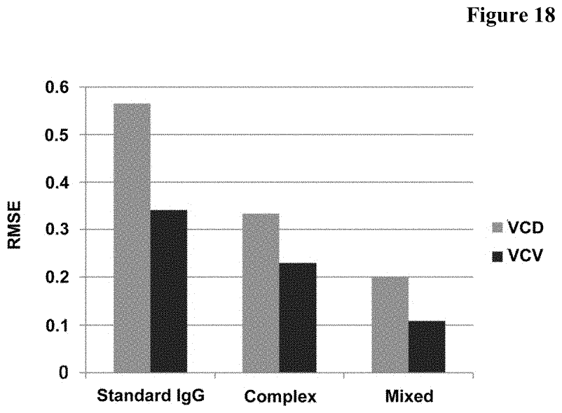

FIG. 19: Behavior of the RMSE considering all models (MLPRegressor, random forest, XGBoost) with the training dataset depending on the target parameter VCV.

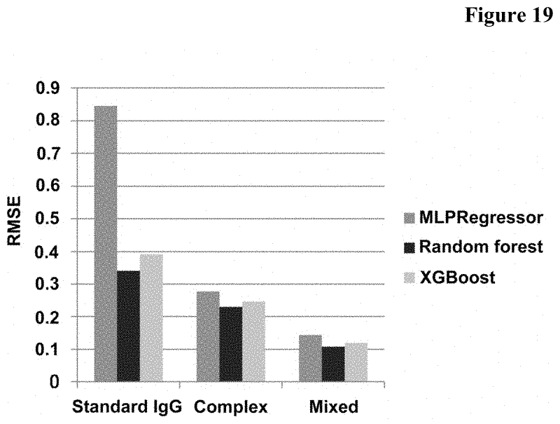

FIG. 20: Bar chart of the difference of the RMSE for the test and training datasets, the best models for the target variable VCV.

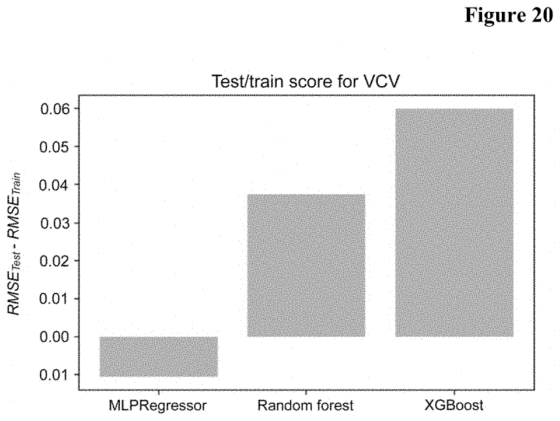

### REFERENCES

[1] J. Glassey, et al., Biotechnol. J. 6 (2011) 369-377.
[2] F. Garcia-Ochoa, et al., Biochem. Eng. J. 49 (2010) 289-307.
[3] E. Trummer, et al., Biotechnol. Bioeng. 94 (2006) 1033-1044.
[4] Y.-M. Huang, et al., Biotechnol. Prog. 26 (2010) 1400-1410.
[5] B C Mulukutla, et al., Met. Eng. 14 (2012) 138-149.
[6] R. Luttmann, et al., Biotechnol. J. 7 (2012) 1040-1048.
[7] T. Becker and D. Krause, Chem. Ing. Tech. 82 (2010) 429-440.
[8] L Z Chen, et al., Bioproc. Biosys. Eng. 26 (2004) 191-195.
[9] P. Kroll, et al., Biotechnol. Lett. 39 (2017) 1667-1673.
[10] S. Raschka and V. Mirjalili, Machine Learning with Python and Scikit-Learn and Tensor-Flow: The comprehensive practice manual for data science, deep learning and predictive analytics. Frechen: mitp, 2., updated and expanded edition, 2018.
[11] Hsu, Chih-Wie, et al., “A practical guide to support vector classification,” Taipei, pp. 1-16, 2003.
[12] R. Kohavi et al., Ijcai, 14 (1995) 1137-1145.
[13] W S McCulloch and W. Pitts, Bull. Math. Biophys. 5 (1943) 115-133.
[14] F. Rosenblatt, Psychol. Rev. 65 (1958) 386-408.
[15] Kriesel David, ed., A brief overview of neural networks.
[16] W. Lu, “Neural network models for distortional buckling behavior of cold-formed steel compression members,” 2000.
[17] R. Rojas, Neural Networks: A Systematic Introduction. Berlin and Heidelberg: Springer, 1996.
[18] L. Breiman, “Random forests,” Machine Learn. 45 (2001) 5-32.
[19] RO Duda, et al., Pattern Classification. sl: Wiley Interscience, 2. Ed., 2012.
[20] L. Breiman, Machine Learn. 24 (1996) 123-140.
[21] Y. Freund and R E Schapire, J. Comp. Syst. Sci. 55: 119-139 (1997).
[22] T. Chen and C. Guestrin, “Xgboost,” in the 22. ACM SIGKDD International Conference (B. Krishnapuram, M. Shah, A. Smola, C. Aggarwal, D. Shen, and R. Rastogi, eds.), pp. 785-794.
[23] L. Fahrmeir, et al., Statistics: The path to data analysis. Springer textbook, Berlin, Heidelberg and s.1.: Springer Berlin Heidelberg, fourth, improved edition ed., 2003.
[24] Kozachenko, L F, et al., Prob. Peredachi Informat. 23 (1987) 9-16.
[25] A. Kraskov, et al., Phys. Rev. E 69 (2004) Pt 2, p. 066138.
[26] BC Ross, PLoS one 9 (2014) e87357.
[27] M. Peleg, J. Sci. Food Agric. 71/2 (1996) 225-230.

### LIST OF ABBREVIATIONS

ambr automated microscale bioreactor
ATP Adenosine triphosphate
Bagging Bootstrap aggregating
CHO Chinese Hamster Ovary
CIP Cleaning in Place
FDA Food and Drug Administration
GMP Good Manufacturing Practice
ANN Artificial Neural Network
MLP Multi Level Perceptron
NADPH Nicotinamide adenine dinucleotide phosphate
OUR Oxygen Uptake Rate
OTR Oxygen Transfer Rate
PAT Process Analytical Technology
RF Random Forest
SIP Sterilization in Place
VCD Viable Cell Density
VCV Viable Cell Volume
XGBoost eXtreme Gradient Boosting

List of Symbols

| Symbol | Description | Unit |
|---|---|---|
| ACO.PV | Exhaust gas CO2 | in % |
| ACOT.PV | Exhaust gas CO2 (total) | in % |
| | Exhaust gas O2 | |
| AO.PV | Chiller temperature | in % |
| CHT.PV | | — |
| CO2.PV | CO2 | in mL/min |
| CO2T.PV | CO2 (total) | in mL/min |
| D | Average diameter | in μm |
| e | Residues | — |
| DRZ.PV | Rotational speed | in rpm |
| fk | Estimation of a decision tree | — |
| FED2T.PV | FEED2 (total) | in mL |
| FED3T.PV | FEED3 (total) | in mL |
| Glucose | Glucose | in mg/L |
| Glucose | Normalized glucose | in AU (Standardized |
| standard | Fermenter volume | values) |
| GEW.PV | | in g |
| K | Number of decision trees | — |
| L | Loss function | — |
| Lactate | Lactate | in mg/L |
| Lactate | Standardized lactate | in AU (Standardized |
| standard | Hydroxide solution | values) |
| LGE.PV | | in mL |
| N2.PV | N2 | in mL/min |
| O | Output neural network | — |
| O2.PV | O2 | in mL/min |
| OUR | OUR | in mol/(L * h) |
| PH.PV | pH | in — |
| PO2.PV | pO2 | in % |
| Process time | Process time | in days |
| (d) | | |
| T | Number of leaves | — |
| TEF.PV | Temperature | in ° C. |
| VCD | in German Live cell density | in 105 cells/mL |
| VCDNorm | Standardized VCD | in AU (standardized |
| | | values) |
| VCV | Viable cell volume | In 10−7 mL (cells)/mL |
| | | (cell suspension) |
| VCDnorm | standardized VCD | in AU (Standardized |
| | | values) |
| w1, . . . , wn | weightings | — |
| X | feature matrix of influencing | — |
| | parameters | |
| x1, . . . , xn | influencing parameters | — |
| y | Actual real target value | — |
| y\hat{ } | Estimated target value | — |
| y | Mean of the real target value | — |
| o | Propagation function | — |
| ϕ | Activation function | — |
| Ω | Regularization term | — |
| λ | Regularization parameter | — |
| γ | Error minimization | — |

### Materials

### Software:

The programming language Python was used in the Spyder development environment for the entire work. The implementation was carried out in object-oriented programming. Several classes were written, which implemented individual tasks within the project.

Software used

| Surname | Function | Version | Company |
|---|---|---|---|
| Spyder | Programming interface | 3.2.8 | Anaconda |
| scikit-learn | Python library for machine | tbd | sklearn |
| | learning | | |
| PI system | Database system | ??? | OSIsoft |
| Spotfire | Visualization tool | 7.7 | TIBCO |
| JMP ® | Stats program | 12.1.0 | SAS |
| Excel ® | Spreadsheet | 2016 | Microsoft |
| notepad ++ | Text editor | 7.5.4 | notepad ++ |

### Methods

### Data Processing

The entire dataset included 155 cultivation runs. These were broken down into on-line and off-line data. Data processing was implemented with Spyder in the Python programming language. The data were available as csv files. The data were read in with the “csv” program library. This allows data to be read in quickly and easily and converted into new data structures within the development environment. A “PlFileParser” class for the on-line data and an “Off-lineDataParser” class for the off-line data have been implemented.

### Interpolation

Since the data were available with different data densities, they had to be interpolated accordingly. A linear interpolation and an interpolation using the moving average method were used for this purpose. Both functions have been implemented with the “scipy” library: “linear-interpolate. interpld” and “moving-average-convolve”. This ensured that the interpolated values were always between two raw measured values. The interpolation is therefore always within the natural fluctuation range of the measurement signal of the process variables. Since each process variable had different time stamps within a file, it was necessary to create another CSV file. The “TimelineMapping” contained all start and end times of the respective cultivation and was created by another database query. Three different intervals were selected for the resolution of the data:
Time stamp of the associated sampling times of the off-line data
1/10 days
Five minutes

Due to the considerably lower data density and the non-linear data course, no linear interpolation was applied to the off-line data. Here three different interpolation strategies were used for fitting:
Peleg fit
Polynomial fit
Spline

The interpolation according to M. Peleg is able to map biological growth through additional functional terms and thus to describe the course of growth well [27]. Therefore, the raw data of the live cell density were fitted with all three interpolations. For glucose and lactate, an interpolation was carried out using the polynomial and spline method, since no biological behavior was to be assumed here. The on-line and off-line datasets were merged for the different intervals and saved as a CSV file for each cultivation. The correlation analysis was then carried out based on these datasets.

### Correlation Analysis

The correlation analysis was carried out with JMP®. With JMP® it is possible to apply statistical analysis to datasets. Multivariate statistics of the on-line data (feature) related to the respective target variable (lactate, glucose, VCD, VCV) was applied. The data are analyzed both for statistical significance in describing the target variables and for linear relationships. The correlation analysis shows linear relationships between an independent and a dependent variable, in the form of the correlation coefficient according to Bravais-Pearson.

### Mutual Information

Another method of identifying suitable features has been used in the form of mutual information. In determination by means of mutual information, the information content is determined, which is contained in an independent variable X in order to describe the target variable Y. The dependencies were calculated and implemented with “sklearn” by means of “mutual information regression”. Based on the size of the datasets with a resolution of five minutes, the information content was calculated separately for each cultivation and then the mean of the values obtained across all cultivations was formed.

### Creation of a Feature Matrix/Results Vector

The creation of the feature matrix came from the result of the correlation analysis and the statistical evaluation based on the information content. This can be represented as a matrix and contains one feature per column and one point in time with the respective version of the feature. The feature matrix was saved as a Panda DataFrame. Thus, a suitable file format was available for training and testing the models.

### Modeling and Evaluation

With the help of the results of the correlation analysis, a separate dataset was created for each target variable. A division of the feature matrix into a training and test dataset was necessary to train the models. A later use for on-line prediction required the withholding of a complete validation project. The training dataset contained 80% and thus 123 cultivation runs of the entire dataset.

Since the prediction of all target variables is a constant target parameter, only regressors were used as models. A number of hyperparameters, which differed from model to model, were available for the models. The training of the models thus served to adapt the hyperparameters so as to map the target variable as precisely as possible.

For the training itself, the entire feature matrix was standardized with the standard scaler of the Scikit-Learn library.

### Optimization of Hyperparameters

The hyperparameters were optimized with a randomized search (RandomizedSearchCV) and a grid-based search (GridSearchCV) from the Scikit-Learn library. All models were trained using the randomized search (RandomizedSearchCV) of the Scikit-Learn library in combination with a tenfold cross-validation of the training dataset. The various areas of the hyperparameters were examined for the smallest RMSE. The randomized search was carried out 30 times. Accordingly, a different, randomly selected set of hyperparameters was used in each iteration. The hyperparameters of the ten models with the smallest RMSE were output. Then, based on the hyperparameters from the randomized search, the hyperparameters for the grid search were more finely graded. The grid search was carried out again with a tenfold cross-validation of the dataset. The model with the least error (smallest RMSE) was saved and then used to estimate the target variables from the test dataset.

### Multilayer Perceptron

The Scikit-Learn library was used to implement the multilayer perceptron (MLP). The following list contains the hyperparameters that were used to train the models:
Number of neurons in the input layer
Number of neurons in the hidden layer
Solver algorithms (adam, lbfgs, sgd) for setting the weights
Activation functions (identity, logistic, tanh, relu)
Learning rate
Maximum number of iterations

### Random Forest

The random forest was also implemented by the Scikit-Learn library. The following candidates were available as hyperparameters within this optimization:
Number of decision trees
Number of features per decision tree
Maximum depth of the decision tree
Minimum number of datasets to create a new node
Methodology for selecting the datasets (bootstrap=true/false)

### XGBoost

The XGBoost algorithm was integrated into the project structure through the XGBoost library. The following hyperparameter space corresponded to:
Number of regression trees in the ensemble
Maximum depth of the regression trees
Learning rater η
Number of datasets per decision tree
Minimum weight of a child node in the decision tree
γ error evaluation
   as the hyperparameters used.

### Model Evaluation

The model evaluation was primarily implemented by displaying an error histogram. This shows the errors (residuals) that the model has when predicting the test dataset for the actual value of the target parameter.

The RMSE was calculated for the accuracy of the estimation of the target parameter and compared with the mean value of the target parameter.

In order to examine the models for overfitting, the RMSE was calculated for the entire training and test dataset. The difference between the two errors was used as an indication of overfitting of the models:

Overfit=RMSEₜₑₛₜ−RMSEₜᵣₐᵢₙ

To further describe the model quality, the coefficient of determination for the entire test dataset and for each cultivation considered individually was used.

### Example 1

### Ambr250-Cultivation

155 datasets based on cultivations in the ambr250 system were collected. The eukaryotic cells used were CHO cells that expressed a target molecule extracellularly. The cultivations were carried out using the fed-batch process. The ambr system used enables twelve cultivations to be carried out simultaneously. The cultivation time of the main culture was 13 to 14 days. The single-use bioreactors (250 mL) provided the reaction space for this. The pre-culturing was carried out in shake flasks and lasted three weeks. The starting conditions in terms of volume and number of cells at the time of inoculation were comparable in each reactor. The media used were exclusively chemically defined media. Only one medium batch was used per cultivation.

In order to provide optimal cultivation conditions within this system, a number of process variables were available. The parameters to be controlled were pH, temperature and the dissolved oxygen concentration in the medium. The following table contains a complete list of all process variables used for this work.

TABLE 12
On-line measured parameters.

| Process variable | PI TAG | Unit |
|---|---|---|
| Exhaust gas CO₂ | ACO.PV | in % |
| Exhaust gas CO₂ | ACOT.PV | in % |
| Exhaust gas O₂ | AO.PV | in % |
| Chiller temperature | CHT.PV | in — |
| CO₂ | CO2.PV | in mL/min |
| CO₂ (total) | CO2T.PV | in mL/min |
| Rotational speed | DRZ.PV | in rpm |
| FEED2 (total) | FED2T.PV | in mL |
| FEED3 (total) | FED3T.PV | in mL |
| Fermenter volume | GEW.PV | in g |
| Hydroxide solution | LGE.PV | in mL |
| N₂ | N2.PV | in mL/min |
| O₂ | O2.PV | In mL/min |
| OUR | OUR | in mol/(L · h) |
| pO₂ | PO2.PV | in % |
| Process time | Process time (d) | in days |
| Temperature | TEF.PV | in ° C. |

All measured variables were recorded over the entire cultivation period by what is termed a PI system. The PI system only contains on-line measured variables.

The parameters listed here were available for monitoring optimal cultivation conditions. Exhaust gas analysis from BlueSens was also available for each reactor. This detects the O₂ and CO₂ content in the exhaust gas flow from the bioreactors and thus provided another important component in the process control. These two measured variables in the exhaust gas flow can be used to determine OUR and OTR.

Samples were taken daily during cultivation. These were then analyzed for various concentrations of the metabolites and product titers using Cedex Bio HAT® (Roche Diagnostics GmbH, Mannheim, Germany).

Furthermore, the cell count measurement was carried out. The measurement provides information about live cell density, total cell density, viability, aggregation rate and cell diameter. These parameters can be used to infer the growth behavior of the culture. The off-line sizes were measured by the Cedex HiRes® (Roche Diagnostics GmbH, Mannheim, Germany) cell counter. The error from these cell counting and cell analysis systems is in a range of 10%. All off-line measured quantities used are shown in the following table.

TABLE 13
Off-line measured variables.

| Process variable | Description | Unit |
|---|---|---|
| Live cell density (Cedex HiRes o_{R}) | VCD | in cells/mL |
| Cell diameter (Cedex HiRes o_{R}) | Avg. | in μm |
| Lactate (Cedex Bio HT o_{R}) | Diameter | |
| Glucose (Cedex Bio HT o_{R}) | Lactate | in mg/L. |
| | glucose | |

What is claimed is:

1. A method for adjusting the glucose concentration to a target value during the mammalian cell cultivation, comprising the following steps
a) determine the current values at least for the process variables ‘Time’, ‘CHT.PV’, ‘ACOT.PV’, ‘FED2T.PV’, ‘GEW.PV’, ‘CO2T.PV’, ‘ACO.PV’, ‘AO.PV’, ‘N2.PV’, ‘CO2.PV’, ‘FED3T.PV’, ‘OUR’ and ‘PH.PV’ in the cultivation,
b) determine the current glucose concentration in the cultivation medium using the measured values from a) by means of a data-driven model for the mammalian cell cultivation, which was generated using a feature matrix comprising the process variables ‘Time’, ‘CHT.PV’, ‘ACOT.PV’, ‘FED2T.PV’, ‘GEW.PV’, ‘CO2T.PV’, ‘ACO.PV’, ‘AO.PV’, ‘N2.PV’, ‘CO2.PV’, ‘FED3T.PV’, ‘OUR’ and ‘PH.PV’, and
c) adding glucose until the target value is reached if the current glucose concentration from b) is lower than the target value, and thus adjusting the glucose concentration to a target value.

2. The method according to claim 1, characterized in that the process variable(s) is/are selected from the group comprising the process variables viable cell density, viable cell volume, glucose concentration in the cultivation medium, and lactate concentration in the cultivation medium.

3. The method according to one of claims 1 through 2, characterized in that the method is carried out without sampling and exclusively using on-line measured values from this cultivation.

4. The method according to one of claims 1 through 3, characterized in that the data-driven model is generated by means of machine learning.

5. The method according to one of claims 1 through 4, characterized in that the data-driven model is generated with the random forest method.

6. The method according to one of claims 1 through 5, characterized in that the data-driven model is generated with a training dataset, which comprises at least 10 cultivation runs.

7. The method according to any one of claims 1 through 6, characterized in that
a) the datasets available for modeling are randomly divided into training and test datasets in a ratio between 70:30 and 80:20,
b) the model is formed,
c) the mean value and the standard deviation for the determination of the process variable for the datasets from the training dataset is determined and the mean value and the standard deviation for the determination of the process for the datasets is determined from the test dataset,
d) steps a) to c) are repeated until comparable mean values and standard deviations with regard to the division between test and training dataset are achieved, wherein the division obtained under a) is different with each new run.

8. The method according to one of claims 1 through 7, characterized in that the datasets, which are used to generate the data-driven model, each contain the same number of data points.

9. The method according to one of claims 1 through 8, characterized in that the data points in the datasets, which are used to generate the data-driven model, are each for the same times of the cultivation.

10. The method according to one of claims 1 through 9, characterized in that missing data points in the datasets are supplemented by interpolation.

11. The method according to claim 10, characterized in that missing data points for the glucose concentration and/or viable cell volume are obtained by a third degree polynomial fit, that missing data points for the lactate concentration are obtained by univariate spline fit, and/or that missing data points for the viable cell density can be obtained through a Peleg fit.

12. The method according to one of claims 1 through 11, characterized in that the datasets contain a data point at least every 144 minutes.

13. The method according to any one of claims 1 through 12, characterized in that the mammalian cell is a CHO-K1 cell.

14. The method according to any one of claims 1 through 13, characterized in that the mammalian cell expresses and secretes an antibody.

15. The method according to any one of claims 1 through 14, characterized in that the data-driven model is generated with a training dataset that contains complex IgG cultivation runs and standard IgG cultivation runs.

16. The method according to any one of claims 1 through 15, characterized in that the cultivation volume is 300 mL or less.
